(19) United States
(12) Patent Application Publication
(10) Pub. No.: US 2019/0076452 A1
(43) Pub. Date: Mar. 14, 2019

(54) THERAPEUTIC COMBINATIONS FOR TREATING NEOPLASIA

(71) Applicant: MEDIMMUNE LIMITED, Cambridge (GB)

(72) Inventors: Jonathan RIOS-DORIA, Gaithersburg, MD (US); ROBERT E. HOLLINGSWORTH, GAITHERSBURG, MD (US)

(21) Appl. No.: 15/525,804

(22) PCT Filed: Nov. 10, 2015
(86) PCT No.: PCT/EP2015/076269

§ 371 Date: May 10, 2017

Related U.S. Application Data
(60) Provisional application No. 62/078,238, filed on Nov. 11, 2014.
(60) Provisional application No. 62/154,287, filed on Apr. 29, 2015.

(51) Int. Cl. A61K 31/704; A61K 45/06; A61K 9/127; A61P 35/00

(52) U.S. Cl. CPC A61K 31/704; A61P 35/00; A61K 9/1271; A61K 45/06

(57) ABSTRACT

The invention features doxorubicin or Doxil in combination with a checkpoint inhibitor (e.g., an anti-CTLA-4 antibody, anti-PD-1 antibody, an anti-PD-L1 antibody, GITR ligand, or OX40 fusion protein (FP) and methods of using the combination to enhance anti-tumor activity in a subject.

### SEQUENCE LISTING

The instant application contains a Sequence Listing which has been submitted electronically in ASCII format and is hereby incorporated by reference in its entirety. Said ASCII copy, created on Oct. 26, 2015, is named IMTC-200WO1_SL.txt and is 24,829 bytes in size.

### BACKGROUND OF THE INVENTION

Cancer continues to be a major global health burden. Despite progress in the treatment of cancer, there continues to be an unmet medical need for more effective and less toxic therapies, especially for those patients with advanced disease or cancers that are resistant to existing therapeutics.

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients.

Two T cell pathways receiving significant attention signal through cytotoxic T lymphocyte antigen-4 (CTLA-4, CD152) and programmed death ligand 1 (PD-L1, also known as B7H-1 or CD274).

CTLA-4 is expressed on activated T cells and serves as a co-inhibitor to keep T cell responses in check following CD28-mediated T cell activation. CTLA-4 is believed to regulate the amplitude of the early activation of naïve and memory T cells following TCR engagement and to be part of a central inhibitory pathway that affects both antitumor immunity and autoimmunity. CTLA-4 is expressed exclusively on T cells, and the expression of its ligands CD8 0 (B7.1) and CD86 (B7.2), is largely restricted to antigen-presenting cells, T cells, and other immune mediating cells. Antagonistic anti-CTLA-4 antibodies that block the CTLA-4 signaling pathway have been reported to enhance T cell activation. One such antibody, ipilimumab, was approved by the FDA in 2011 for the treatment of metastatic melanoma. Another anti-CTLA-4 antibody, tremelimumab, was tested in phase III trials for the treatment of advanced melanoma, but did not significantly increase the overall survival of patients compared to the standard of care (temozolomide or dacarbazine) at that time.

PD-L1 is also part of a complex system of receptors and ligands that are involved in controlling T cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1-dependent immunosuppressive effects and enhance the cytotoxic activity of antitumor T cells in vitro. MEDI4736 is a human monoclonal antibody directed against human PD-L1 that is capable of blocking the binding of PD-L1 to both the PD-1 and CD80 receptors.

Despite the significant progress made over the past decade in developing strategies for combatting cancer and other diseases, patients with advanced, refractory and metastatic disease have limited clinical options. Chemotherapy, irradiation, and high dose chemotherapy have become dose limiting. There remains a substantial unmet need for new less-toxic methods and therapeutics that have better therapeutic efficacy, longer clinical benefit, and improved safety profiles, particularly for those patients with advanced disease or cancers that are resistant to existing therapeutics.

### SUMMARY OF THE INVENTION

As described below, the present invention features doxorubicin or Doxil in combination with an immunomodulatory agent (e.g., an anti-CTLA-4 antibody, anti-PD-1 antibody, an anti-PD-L1 antibody, GITR ligand, or OX40 fusion protein (FP)) and methods of using the combination to enhance anti-tumor activity in a subject.

In one aspect, the invention provides a method of increasing anti-tumor activity in a subject, the method involving administering doxorubicin or a polyethylene glycol coated liposome encapsulated form of doxorubicin and an immunomodulatory agent that is one or more of an anti-PD-1 antibody, anti-PD-L1 antibody, anti-CTLA-4 antibody, a glucocorticoid-induced TNFR-related gene (GITR) ligand, and an OX40 fusion protein to a subject.

In another aspect, the invention provides a method of increasing an anti-tumor immune response in a subject, the method involving administering doxorubicin or a polyethylene glycol coated liposome encapsulated form of doxorubicin and an immunomodulatory agent that is one or more of an anti-PD-1 antibody, anti-PD-L1 antibody, anti-CTLA-4 antibody, a glucocorticoid-induced TNFR-related gene (GITR) ligand, and an OX40 fusion protein to a subject.

In another aspect, the invention provides a method of treating a tumor in a subject, the method involving administering doxorubicin or a polyethylene glycol coated liposome encapsulated form of doxorubicin and an immunomodulatory agent that is one or more of an anti-PD-1 antibody, anti-PD-L1 antibody, anti-CTLA-4 antibody, a glucocorticoid-induced TNFR-related gene (GITR) ligand, and an OX40 fusion protein to a subject.

In one aspect, the invention provides a kit for increasing anti-tumor activity, the kit containing doxorubicin or a polyethylene glycol coated liposome encapsulated form of doxorubicin (e.g., Doxil) and an immunomodulatory agent that is one or more of an anti-PD-1 antibody, anti-PD-L1 antibody, anti-CTLA-4 antibody, a GITR ligand, and an OX40 agonist. In some embodiments, the kit includes instructions for using the kit according to the methods of the invention.

In another aspect, the invention provides a pharmaceutical formulation containing an effective amount of doxorubicin or a polyethylene glycol coated liposome encapsulated form of doxorubicin (e.g., Doxil) and an effective amount of an immunomodulatory agent that is one or more of an anti-PD-1 antibody, anti-PD-L1 antibody, anti-CTLA-4 antibody, a GITR ligand, and an OX40 agonist.

In various embodiments of any aspect delineated herein, the polyethylene glycol coated liposome encapsulated form of doxorubicin is Doxil®.

In various embodiments of any aspect delineated herein, the anti-PD-L1 antibody is MEDI4736, BMS-936559, or MPDL3280A. In particular embodiments, the anti-PD-L1 antibody is MEDI4736.

In various embodiments of any aspect delineated herein, the anti-PD-1 antibody is LOPD 18, nivolumab, pembrolizumab, lambrolizumab, MK-3475, AMP-224, and pidilizumab. IN particular embodiments, the anti-PD-1 antibody is LOPD 18.

In various embodiments of any aspect delineated herein, the anti-CTLA-4 antibody is tremelimumab or ipilimumab. In particular embodiments, the anti-CTLA-4 antibody is tremelimumab.

In various embodiments of any aspect delineated herein, the immunomodulatory agent is a GITR ligand or GITR ligand fusion protein.

In various embodiments of any aspect delineated herein, the immunomodulatory agent is an OX40 fusion protein.

In various embodiments of any aspect delineated herein, the tumor is a colon carcinoma or sarcoma.

In various embodiments of any aspect delineated herein, the method results in an increase in overall survival as compared to the administration of any one of doxorubicin, a polyethylene glycol coated liposome encapsulated form of doxorubicin (e.g., Doxil), an anti-PD-1 antibody, anti-PD-L1 antibody, anti-CTLA-4 antibody, a GITR ligand, and an OX40 fusion protein alone. In various embodiments, the method induces a tumor-specific immune response.

In various embodiments of any aspect delineated herein, doxorubicin or polyethylene glycol coated liposome encapsulated form of doxorubicin (e.g., Doxil) is administered in combination with an anti-PD-1 antibody, including any one or more of LOPD 18, nivolumab, pembrolizumab, lambrolizumab, MK-3475, AMP-224, and pidilizumab

In various embodiments of any aspect delineated herein, doxorubicin or polyethylene glycol coated liposome encapsulated form of doxorubicin (e.g., Doxil) is administered in combination with an anti-PD-L1 antibody, including any one or more of MEDI4736, BMS-936559, and MPDL3280A.

In various embodiments of any aspect delineated herein, doxorubicin or polyethylene glycol coated liposome encapsulated form of doxorubicin (e.g., Doxil) is administered in combination with an anti-CTLA-4 antibody, including any or more of tremelimumab and ipilimumab.

In various embodiments of any aspect delineated herein, doxorubicin or polyethylene glycol coated liposome encapsulated form of doxorubicin (e.g., Doxil) is administered in combination with a GITR ligand or GITR ligand fusion protein.

In various embodiments of any aspect delineated herein, doxorubicin or polyethylene glycol coated liposome encapsulated form of doxorubicin (e.g., Doxil) is administered in combination with an OX40 fusion protein.

In various embodiments of any aspect delineated herein, the administration of doxorubicin, polyethylene glycol coated liposome encapsulated form of doxorubicin (e.g., Doxil), or the immunomodulatory agent (e.g., an anti-PD-1 antibody, anti-PD-L1 antibody, anti-CTLA-4 antibody, a GITR ligand, or an OX40 fusion protein) is by intravenous infusion.

In various embodiments of any aspect delineated herein, doxorubicin or polyethylene glycol coated liposome encapsulated form of doxorubicin (e.g., Doxil) and the immunomodulatory agent are administered concurrently.

In various embodiments of any aspect delineated herein, doxorubicin or polyethylene glycol coated liposome encapsulated form of doxorubicin (e.g., Doxil) is administered prior to the administration of the immunomodulatory agent.

In various embodiments of any aspect delineated herein, the immunomodulatory agent is administered prior to the administration of the polyethylene glycol coated liposome encapsulated form of doxorubicin (e.g., Doxil).

In various embodiments of any aspect delineated herein, the subject is a human patient.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “anti-tumor activity” is meant any biological activity that reduces or stabilizes the proliferation or survival of a tumor cell. In one embodiment, the anti-tumor activity is an anti-tumor immune response.

By “immunomodulatory agent” is meant an agent that enhances an immune response (e.g., anti-tumor immune response). Exemplary immunomodulatory agents of the invention include antibodies, such as an anti-CTLA-4 antibody, anti-PD-1 antibody, an anti-PD-L1 antibody, and fragments thereof, as well as proteins, such as GITR ligand, or OX40 fusion protein, or fragments thereof. In one embodiment, the immunomodulatory agent is an immune checkpoint inhibitor.

By “PD-1 polypeptide” is meant a polypeptide or fragment thereof having at least about 85% amino acid identity to NCBI Accession No. NP_005009 and having PD-L1 and/or PD-L2 binding activity. The sequence of NP_005009 is provided below.

| (SEQ ID NO: 1) | |
|---|---|
| 1 | mqipqapwpv vwavlqlgwr pgwfldspdr pwnpptfspa llvvtegdna tftcsfsnts |
| 61 | esfvlnwyrm spsnqtdkla afpedrsqpg qdcrfrvtql pngrdfhmsv vrarrndsgt |
| 121 | ylcgaislap kaqikeslra elrvterrae vptahpspsp rpagqfqtlv vgvvggllgs |
| 181 | lvllvwvlav icsraargti garrtgqplk edpsavpvfs vdygeldfqw rektpeppvp |
| 241 | cvpeqteyat ivfpsgmgts sparrgsadg prsaqplrpe dghcswpl |

By “PD-1 nucleic acid molecule” is meant a polynucleotide encoding a PD-1 polypeptide. An exemplary PD-1 nucleic acid molecule sequence is provided at NCBI Accession No. NM_005018.

By “anti-PD-1 antibody” is meant an antibody that selectively binds a PD-1 polypeptide. LOPD 180 is an exemplary PD-1 antibody.

LOPD180 heavy chain variable region polypeptide
sequence
LOPD180_VH_AA
(SEQ ID NO: 2)
QVQLQESGPGLVKPSQTLSLTCTVSGGSISSGAYYWSWIRQHPGKGLEWI
GYIYYNGNTYYNPSLRSRVTISVDTSKNQFSLKLSSVTAADTAVYYCVRA
SDYVWGGYHYFDAFDLWGRGTLVTVSS
LOPD180 heavy chain variable region nucleic acid
sequence
LOPD180_VH_DNA
(SEQ ID NO: 3)
CAGGTGCAGCTGCAGGAGTCGGGCCCAGGACTGGTGAAGCCTTCACAGAC
CCTGTCCCTCACCTGCACTGTCTCTGGTGGCTCCATCAGCAGTGGTGCTT
ATTACTGGAGCTGGATCCGCCAGCACCCAGGGAAGGGCCTGGAGTGGATT
GGGTACATCTATTACAATGGGAACACGTACTACAACCCGTCCCTCAGGAG
TCGAGTTACCATATCAGTAGACACGTCTAAGAACCAGTTCTCCCTGAAGC
TGAGCTCTGTGACTGCCGCGGACACGGCCGTCTATTACTGTGTGAGAGCG
TCTGATTACGTTTGGGGGGGTTATCATTATTTCGACGCGTTCGACCTCTG
GGGCCGGGGAACCCTGGTCACCGTCTCCTCA
LOPD180 light chain variable region polypeptide
sequence
LOPD180_VL_AA
(SEQ ID NO: 4)
QSVLTQPPSASGTPGQRVTISCSGSNSNIGSNSVNWYQQLPGTAPKLLIY
GNNQRPSGVPDRFSGSKSGTSASLAISGLQSEDEADYYCAAWDDSLNGPV
FGGGTKVTVL
LOPD180 light chain variable region nucleic acid
sequence
LOPD180_VL_DNA
(SEQ ID NO: 5)
CAGTCTGTGCTGACTCAGCCACCCTCAGCGTCTGGGACCCCCGGGCAGAG
GGTCACCATCTCTTGTTCTGGAAGCAACTCCAACATCGGAAGTAATTCTG
TAAACTGGTACCAGCAGCTCCCAGGAACGGCCCCCAAACTCCTCATCTAT
GGTAATAATCAGCGGCCCTCAGGGGTCCCTGACCGATTCTCTGGCTCCAA
GTCTGGCACCTCAGCCTCCCTGGCCATCAGTGGGCTCCAGTCTGAGGATG
AGGCTGATTATTACTGTGCAGCATGGGATGACAGCCTGAATGGTCCGGTA
TTCGGCGGAGGGACCAAGGTCACCGTCCTA

Other exemplary anti-PD-1 antibodies include nivolumab (ONO-4538/BMS-936558 or MDX110, Opdivo; BMS; approved), pembrolizumab (Keytrudat®, lambrolizumab, MK-3475; Merck; approved), AMP-224 (Amplimmune/GSK), and pidilizumab (CT-011; Teva/Curetech).

By “PD-L1 polypeptide” is meant a polypeptide or fragment thereof having at least about 85% amino acid identity to NCBI Accession No. NP_001254635 and having PD-1 and CD80 binding activity.

By “PD-L1 nucleic acid molecule” is meant a polynucleotide encoding a PD-L1 polypeptide. An exemplary PD-L1 nucleic acid molecule sequence is provided at NCBI Accession No. NM_001267706.

By “anti-PD-L1 antibody” is meant an antibody that selectively binds a PD-L1 polypeptide. Exemplary anti-PD-L1 antibodies are described for example at US20130034559/U.S. Pat. No. 8,779,108 and US20140356353, which is herein incorporated by reference. MEDI4736 is an exemplary anti-PD-L1 antibody. Other anti-PD-L1 antibodies include BMS-936559 (Bristol-Myers Squibb) and MPDL3280A (Roche).

MEDI4736 VL
(SEQ ID NO: 6)
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
MEDI4736 VH
(SEQ ID NO: 7)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
MEDI4736 VH CDR1
(SEQ ID NO: 8)
RYWMS
MEDI4736 VH CDR2
(SEQ ID NO: 9)
NIKQDGSEKYYVDSVKG
MEDI4736 VL CDR1
(SEQ ID NO: 10)
RASQRVSSSYLA
MEDI4736 VL CDR2
(SEQ ID NO: 11)
DASSRAT
MEDI4736 VL CDR3
(SEQ ID NO: 12)
QQYGSLPWT

By “CTLA-4 polypeptide” is meant a polypeptide having at least 85% amino acid sequence identity to GenBank Accession No. AAL07473.1 or a fragment thereof having T cell inhibitory activity. The sequence of AAL07473.1 is provided below:

gi|15778586|gb|AAL07473.1|AF414120_1 CTLA-4
[Homo sapiens]
(SEQ ID NO: 13)
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

By “CTLA-4 nucleic acid molecule” is meant a polynucleotide encoding a CTLA-4 polypeptide. An exemplary CTLA-4 polynucleotide is provided at GenBank Accession No. AAL07473.

By “anti-CTLA-4 antibody” is meant an antibody that selectively binds a CTLA-4 polypeptide. Exemplary anti-CTLA-4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA-4 antibody. Tremelimumab sequences are provided below.

Tremelimumab U.S. Pat. No. 6,682,736

Tremelimumab VL
(SEQ ID NO: 14)
PSSLSASVGDRVTITCRASQSINSYLDWYQQKPGKAPKLLIYAASSLQSG
VPSRFSGSGSGTDFTLTISSLQPEDFATYYCQQYYSTPFTFGPGTKVEIK
RTVAAPSVFIFPPSDEQLKSGTASVVCLLNNFYPREAKV
Tremelimumab VH
(SEQ ID NO: 15)
GVVQPGRSLRLSCAASGFTFSSYGMHWVRQAPGKGLEWVAVIWYDGSNKY
YADSVKGRFTISRDNSKNTLYLQMNSLRAEDTAVYYCARDPRGATLYYYY
YGMDVWGQGTTVTVSSASTKGPSVFPLAPCSRSTSESTAALGCLVKDYFP
EPVTVSWNSGALTSGVH
Tremelimumab VH CDR1
(SEQ ID NO: 16)
GFTFSSYGMH
Tremelimumab VH CDR2
(SEQ ID NO: 17)
VIWYDGSNKYYADSV
Tremelimumab VH CDR3
(SEQ ID NO: 18)
DPRGATLYYYYYGMDV
Tremelimumab VL CDR1
(SEQ ID NO: 19)
RASQSINSYLD
Tremelimumab VL CDR2
(SEQ ID NO: 20)
AASSLQS
Tremelimumab VL CDR3
(SEQ ID NO: 21)
QQYYSTPFT

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind, for example, CTLA-4, PD-1, or PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “doxorubicin” is meant a small compound having the following structural formula:

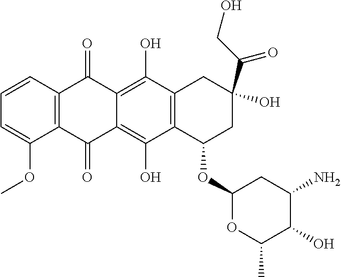 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

CAS 23214-92-8, which is sold under the trade name Adriamycin. Doxil® is the trade name for a polyethylene glycol coated liposome encapsulated form of doxorubicin, which is available from Janssen Products LP.

By “glucocorticoid-induced TNFR-related gene (GITR) polypeptide” is meant a protein having at least about 85% amino acid sequence identity to NP_683699 or a fragment there of that and having T cell regulatory activity. In one embodiment, GITR regulates T cell survival.

| (SEQ ID NO: 22) | |
|---|---|
| 1 | maqhgamgaf ralcglallc alslgqrptg gpgcgpgrll lgtgtdarcc rvhttrccrd |
| 61 | ypgeeccsew dcmcvqpefh cgdpccttcr hhpcppgqgv qsqgkfsfgf qcidcasgtf |
| 121 | sggheghckp wtdccwrcrr rpktpeaass prksgasdrq rrrggwetcg cepgrppgpp |
| 181 | taaspspgap qaagalrsal grallpwqqk wvqeggsdqr pgpcssaaaa gperreretq |
| 241 | swppsslagp dgvgs |

GITR is also termed Tumor Necrosis Factor Receptor Superfamily, Member 18.

By “glucocorticoid-induced TNFR-related gene (GITR) ligand” is meant a protein or fragment thereof that specifically binds GITR and has at least about 85% amino acid sequence identity to NP_005083. The sequence of NP_005083, an exemplary human GITR ligand, is provided below:

| (SEQ ID NO: 23) | |
|---|---|
| 1 | mtlhpspitc eflfstalis pkmclshlen mplshsrtqg aqrsswklwl fcsivmllfl |
| 61 | csfswlifif lqletakepc makfgplpsk wqmasseppc vnkvsdwkle ilqnglyliy |
| 121 | gqvapnanyn dvapfevrly knkdmiqtlt nkskiqnvgg tyelhvgdti dlifnsehqv |
| 181 | lknntywgii llanpqfis |

In one embodiment, a GITR ligand is a GITR agonist or GITR ligand fusion protein. GITR agonists bind GITR and induce tumor regression. GITR ligands are described, for example, by Clothier et al., The Journal of Immunology Oct. 3, 2014 1401002.

By “OX40 fusion protein” is meant a protein that specifically binds the OX40 receptor and increases an immune response. In one embodiment, binding of an OX40 fusion protein to the OX-40 receptor enhances a tumor antigen specific immune response by boosting T-cell recognition. Exemplary OX40 fusion proteins are described in U.S. Pat. No. 7,959,925, entitled, “Trimeric OX40 Immunoglobulin Fusion Protein and Methods of Use.” See, for example, U.S. Pat. No. 7,959,925, SEQ ID NO. 8:

(SEQ ID NO: 24)
LATDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGKELLGGGSIKQIEDKIEEILS
KIYHIENEIARIKKLIGERGHGGGSNSQVSHRYPRFQSIKVQFTEYKKEK
GFILTSQKEDEIMKVQNNSVIINCDGFYLISLKGYFSQEVNISLHYQKDE
EPLFQLKKVRSVNSLMVASLTYKDKVYLNVTTDNTSLDDFHVNGGELILI
HQNPGEFCVL

Other OX40 fusion proteins are described, for example, in U.S. Pat. No. 6,312,700. In one embodiment, an OX40 fusion protein enhances tumor-specific T-cell immunity.

By “reference” is meant a standard of comparison.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1J are graphs showing synergy of doxorubicin or Doxil in combination with α-PD-1 and α-CTLA-4 antibodies in the CT26 tumor model. FIG. 1A is a graph depicting tumor volume in untreated mice. FIG. 1B is a graph depicting tumor volume in mice administered Isotype Controls (rat IgG2a+mouse IgG2b (5/0.5 mg/kg). FIG. 1C is a graph depicting tumor volume in mice administered doxorubicin (4 mg/kg). FIG. 1D is a graph depicting tumor volume in mice administered Doxil (1 mg/kg). FIG. 1E is a graph depicting tumor volume in mice administered α-PD-1 (5 mg/kg). FIG. 1F is a graph depicting tumor volume in mice administered α-CTLA-4 (0.5 mg/kg). FIG. 1G is a graph depicting tumor volume in mice administered doxorubicin+α-PD-1 (4/5 mg/kg). FIG. 1H is a graph depicting tumor volume in mice administered Doxil+α-PD-1 (1/5 mg/kg). FIG. 1I is a graph depicting tumor volume in mice administered doxorubicin+α-CTLA-4 (4/0.5 mg/kg), FIG. 1J is a graph depicting tumor volume in mice administered Doxil+α-CTLA-4 (1/0.5 mg/kg). *, p<0.005 (Bliss independence test). CT26 cells were implanted into Balb/C mice. Four days after cell implantation, mice were randomized by body weight and dosed with Doxil on Day 4, 11, and 17; doxorubicin on Day 4, 8 and 12; and anti-PD-1 or anti-CTLA-4 on Days 10, 14, 17 and 21.

FIGS. 2A and 2B show the survival of mice treated with Doxil or doxorubicin alone or in combination with α-PD-1 and α-CTLA-4. The survival of mice from the study in FIGS. 1A-1J is shown. FIG. 2A is a graph showing survival of the groups of mice administered α-PD-1 in combination with Doxil or doxorubicin, and related control groups. FIG. 2B is a graph showing survival of the groups of mice administered α-CTLA-4 alone or in combination with Doxil or doxorubicin, and related control groups. Mice treated with α-PD-1 or α-CTLA-4 antibodies in combination with Doxil (1 mg/kg) or Doxorubicin (4 mg/kg) survived longer than mice treated with either α-PD-1 or α-CTLA-4 alone. The α-PD-1+doxorubicin (*) and α-CTLA-4+doxorubicin (#) groups were statistically different compared to the doxorubicin group (p=0.005 and p=0.0012 by the Log-rank test, respectively).

FIGS. 3A-3D show that mice achieving complete response with Doxil alone or in combination with anti-CTLA-4 or anti-PD-1 antibodies resisted tumor-rechallenge. FIG. 3A is a graph depicting tumor volume in naïve Balb/C mice (n=10). FIG. 3B is a graph depicting tumor volume in mice that achieved complete response by Doxil treatment and re-challenged with CT26 cells. FIG. 3C is a graph depicting tumor volume in mice that achieved complete response by α-CTLA-4+Doxil treatment (n=10) and re-challenged with CT26 cells. FIG. 3D is a graph depicting tumor volume in mice that achieved complete response by α-PD-1+Doxil treatment (n=9) and re-challenged with CT26 cells. The numbers indicate the number of mice that rejected tumors of the total number of mice in the group.

FIGS. 4A-4E show that T cells are required for Doxil activity in vivo. FIG. 4A is a graph showing tumor volume in CT26 tumor-bearing athymic nude mice dosed with Doxil (5 mg/kg) or Doxorubicin (5 mg/kg) as indicated. FIG. 4B is a graph showing tumor volume in CT26 tumor-bearing Balb/C mice dosed with Doxil (5 mg/kg) or Doxorubicin (5 mg/kg) as indicated. FIG. 4C is a graph showing tumor volume in CT26 tumor-bearing athymic nude mice dosed with gemcitabine (75 mg/kg) as indicated. FIG. 4D is a graph showing tumor volume in CT26 tumor-bearing Balb/C mice dosed with gemcitabine (75 mg/kg) as indicated. FIG. 4E is a graph showing tumor volume in CT26 tumor-bearing athymic nude mice dosed with oxiplatin (8 mg/kg) as indicated. FIG. 4F is a graph showing tumor volume in CT26 tumor-bearing Balb/C mice dosed with oxiplatin (8 mg/kg) as indicated. Arrows indicate dose administration.

FIGS. 5A-5L show synergistic anti-tumor responses of Doxil in combination with multiple immunotherapies in an established CT26 tumor model. FIG. 5A is a graph depicting tumor volume in untreated mice. FIG. 5B is a graph depicting tumor volume in mice administered Doxil. FIG. 5C is a graph depicting tumor volume in mice administered OX40L fusion protein (FP). FIG. 5D is a graph depicting tumor volume in mice administered α-PD-1. FIG. 5E is a graph depicting tumor volume in mice administered α-PD-L1. FIG. 5F is a graph depicting tumor volume in mice administered α-CTLA-4. FIG. 5G is a graph depicting tumor volume in mice administered GITR ligand fusion protein (GITRL FP). FIG. 5H is a graph depicting tumor volume in mice administered Doxil+OX40L FP. FIG. 5I is a graph depicting tumor volume in mice administered Doxil+α-PD-1. FIG. 5J is a graph depicting tumor volume in mice administered Doxil+α-PD-L1. FIG. 5K is a graph depicting tumor volume in mice administered Doxil+α-CTLA-4. FIG. 5L is a graph depicting tumor volume in mice administered Doxil+GITRL FP. Balb/C mice bearing established (˜200-300 mm3) CT26 tumors were randomized by tumor volume and treated with maximally efficacious doses of Doxil (5 mg/kg, Day 11 and 19); OX40L FP (2.5 mg/kg, Day 14 and 19); α-PD-1 (20 mg/kg, Day 11, 14, 19, and 22); α-PD-L1, (30 mg/kg; Day 11, 14, 19, and 22); α-CTLA-4 (20 mg/kg, Day 14, 19, 22, and 26) and GITRL FP (5 mg/kg Day 14, 19, 22, 26, 29 and 32). The CR number indicates the number of mice that achieved complete response out of 12. #, p=0.056; *, p<0.008, Bliss independence test.

FIGS. 6A-6E show survival of mice in a CT26 established-tumor study. The survival of mice from the CT26 established tumor study in FIGS. 5A-5L is shown. FIG. 6A is a graph showing survival of the groups of mice administered OX40 FP alone or in combination with Doxil, and related control groups. FIG. 6B is a graph showing survival of the groups of mice administered α-PD-1 alone or in combination with Doxil, and related control groups. FIG. 6C is a graph showing survival of the groups of mice administered α-PD-L1 alone or in combination with Doxil, and related control groups. FIG. 6D is a graph showing survival of the groups of mice administered α-CTLA-4 alone or in combination with Doxil, and related control groups. FIG. 6E is a graph showing survival of the groups of mice administered GITRL FP alone or in combination with Doxil, and related control groups. *p<0.00625 and statistically significant compared to single-agent therapy by the Log-rank test. #, p<0.00625 and statistically significant compared to Doxil treatment by the Log-rank test.

FIGS. 7A-7L show synergistic anti-tumor responses of Doxil in combination with α-PD-1, α-PD-L1 and α-CTLA-4 antibodies in the MCA205 syngeneic model. FIG. 7A is a graph depicting tumor volume in untreated mice. FIG. 7B is a graph depicting tumor volume in mice administered Doxil. FIG. 7C is a graph depicting tumor volume in mice administered OX40L fusion protein (FP). FIG. 7D is a graph depicting tumor volume in mice administered α-PD-1. FIG. 7E is a graph depicting tumor volume in mice administered α-PD-L1. FIG. 7F is a graph depicting tumor volume in mice administered α-CTLA-4. FIG. 7G is a graph depicting tumor volume in mice administered GITR ligand fusion protein (GITRL FP). FIG. 7H is a graph depicting tumor volume in mice administered Doxil+OX40L FP. FIG. 7I is a graph depicting tumor volume in mice administered Doxil+α-PD-1. FIG. 7J is a graph depicting tumor volume in mice administered Doxil+α-PD-L1. FIG. 7K is a graph depicting tumor volume in mice administered Doxil+α-CTLA-4. FIG. 7L is a graph depicting tumor volume in mice administered Doxil+GITRL FP. C57/Bl6 mice bearing established (˜100-150 mm³) MCA205 tumors were randomized by tumor volume and treated with maximally efficacious doses of Doxil (5 mg/kg, Day 10, 17, and 24); OX40L FP (20 mg/kg, Day 10 and 14); α-PD-1 (10 mg/kg, Day 10, 14, 17 and 21); α-PD-L1, (20 mg/kg; Day 10, 14, 17 and 21); α-CTLA-4 (10 mg/kg, Day 10, 14, 17 and 21) and GITRL FP (5 mg/kg Day 10, 14, 17, 21, 24 and 28). The CR number indicates the number of mice that achieved complete response out of 12. *p<0.008, Bliss independence test.

FIGS. 8A-8E show survival of mice in an MCA205 established-tumor study. The survival of mice from the MCA205 established tumor study in FIGS. 7A-7L is shown. FIG. 8A is a graph showing survival of the groups of mice administered OX40 FP alone or in combination with Doxil, and related control groups. FIG. 8B is a graph showing survival of the groups of mice administered α-PD-1 alone or in combination with Doxil, and related control groups. FIG. 8C is a graph showing survival of the groups of mice administered α-PD-L1 alone or in combination with Doxil, and related control groups. FIG. 8D is a graph showing survival of the groups of mice administered α-CTLA-4 alone or in combination with Doxil, and related control groups. FIG. 8E is a graph showing survival of the groups of mice administered GITRL alone or in combination with Doxil, and related control groups.

FIGS. 9A-9I show that Doxil has immunomodulatory functions of in vivo. MCA205 tumor-bearing C57/Bl6 mice were dosed with α-PD-L1, Doxil, or the combination as herein. FIG. 9A is a graph depicting the percent of CD8⁺ T cells in the blood. FIG. 9B is a graph depicting the percent of CD8⁺ T cells in the tumor. FIG. 9C is a graph depicting the percent of CD4⁺/FoxP3⁺ cells in the tumor. FIG. 9D is a graph depicting expression of CD80 in CD45⁺CD11c⁺MHCII^{hi} cells in the blood. FIG. 9E is a graph depicting expression of CD80 in CD45⁺CD11c⁺MHCII^{hi} cells in the tumor. FIG. 9F is a graph depicting that the percent of CD45⁺CD11c⁺MHCII^{hi} cells was increased in the blood in Doxil treated animals, which was further augmented by the addition of α-PD-L1. FIG. 9G is a graph depicting expression of CD80 in tumor-isolated CD45⁺CD11b⁺Ly6C⁺ cells. FIG. 9H is a graph depicting expression of CD80 in tumor-isolated CD45⁺CD11b⁺Ly6G⁺ cells. FIG. 9I is a graph depicting that the percent of CD45⁺CD11b⁺Ly6C⁺ cells was increased in the tumor in Doxil and Doxil+α-PD-L1 treated animals. *p<0.05, **p<0.01 (unpaired two-tailed Student's t test).

### DETAILED DESCRIPTION OF THE INVENTION

As described below, the present invention features doxorubicin or Doxil in combination with an immunomodulatory agent (e.g., an anti-CTLA-4 antibody, anti-PD-1 antibody, an anti-PD-L1 antibody, GITRL, or OX40 fusion protein (FP)).

Doxorubicin is a widely-used chemotherapeutic drug for patients with sarcoma, lung, breast and other cancers. Previously, doxorubicin has been well-characterized as a DNA intercalator and an inhibitor of topoisomerase. Other mechanisms of action of doxorubicin that are reported are DNA cross-linking, interference with DNA strand separation, free-radical formation, helicase activity and direct membrane effects. Doxorubicin thus has been viewed as a cytotoxic agent with direct cell-killing effects on tumor cells. More recently, doxorubicin has been established as an inducer of immunogenic cell death and has been shown to increase IFN gamma production, and induce dendritic and T cell tumor infiltration in mouse models.

As described herein, both doxorubicin and Doxil synergized with several T-cell targeted immunotherapies in two syngeneic mouse models. Importantly, combination activity was long lasting, leading to high cure rates, and generated immunological memory in the mouse models. Furthermore, the results reveal for the first time that Doxil has direct effects on dendritic and immature myeloid cells in tumors following systemic administration.

CTLA-4, PD-1 and PD-L1

There is mounting evidence that T cells control tumor growth and survival in cancer patients, both in early and late stages of the disease. However, tumor-specific T-cell responses are difficult to mount and sustain in cancer patients.

Two T cell modulatory pathways receiving significant attention signal through cytotoxic T lymphocyte antigen-4 (CTLA-4, CD152) and programmed death ligand 1 (PD-L1, also known as B7H-1 or CD274).

CTLA-4 is expressed on activated T cells and serves as a co-inhibitor to keep T cell responses in check following CD28-mediated T cell activation. CTLA-4 is believed to regulate the amplitude of the early activation of naïve and memory T cells following TCR engagement and to be part of a central inhibitory pathway that affects both antitumor immunity and autoimmunity. CTLA-4 is expressed on T cells, and the expression of its ligands CD80 (B7.1) and CD86 (B7.2), is largely restricted to antigen-presenting cells, T cells, and other immune mediating cells. Antagonistic anti-CTLA-4 antibodies that block the CTLA-4 signaling pathway have been reported to enhance T cell activation. One such antibody, ipilimumab, was approved by the FDA in 2011 for the treatment of metastatic melanoma. Another anti-CTLA-4 antibody, tremelimumab, was tested in phase III trials for the treatment of advanced melanoma but did not significantly increase the overall survival of patients compared to the standard of care (temozolomide or dacarbazine) at that time.

PD-L1 is also part of a complex system of receptors and ligands that are involved in controlling T cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors (e.g., PD-1) are able to relieve PD-L1-dependent immunosuppressive effects and enhance the cytotoxic activity of antitumor T cells in vitro.

PD-1 is a 50-55 kDa type I transmembrane receptor that was originally identified in a T cell line undergoing activation-induced apoptosis. PD-1 is expressed on T cells, B cells, and macrophages. The ligands for PD-1 are the B7 family members PD-L1 (B7-H1) and PD-L2 (B7-DC).

PD-1 is a member of the immunoglobulin (Ig) superfamily that contains a single Ig V-like domain in its extracellular region. The PD-1 cytoplasmic domain contains two tyrosines, with the most membrane-proximal tyrosine (VAYEEL (SEQ ID NO: 25) in mouse PD-1) located within an ITIM (immuno-receptor tyrosine-based inhibitory motif). The presence of an ITIM on PD-1 indicates that this molecule functions to attenuate antigen receptor signaling by recruitment of cytoplasmic phosphatases. Human and murine PD-1 proteins share about 60% amino acid identity with conservation of four potential N-glycosylation sites, and residues that define the Ig-V domain. The ITIM in the cytoplasmic region and the ITIM-like motif surrounding the carboxy-terminal tyrosine (TEYATI (SEQ ID NO: 26) in human and mouse) are also conserved between human and murine orthologues.

PD-1 is expressed on activated T cells, B cells, and monocytes. Experimental data implicates the interactions of PD-1 with its ligands in downregulation of central and peripheral immune responses. In particular, proliferation in wild-type T cells but not in PD-1-deficient T cells is inhibited in the presence of PD-L1. Additionally, PD-1-deficient mice exhibit an autoimmune phenotype. PD-1 deficiency in the C57BL/6 mice results in chronic progressive lupus-like glomerulonephritis and arthritis. In Balb/c mice, PD-1 deficiency leads to severe cardiomyopathy due to the presence of heart-tissue-specific self-reacting antibodies.

Anti-PD-1 and Anti-PD-L1 Antibodies

Anti-PD-1 antibodies and their antigen-binding fragments have been described (see e.g., U.S. Pat. No. 7,488,802, which is herein incorporated by reference in its entirety). LOPD180 is an exemplary PD-1 antibody. Antibodies that specifically bind and inhibit PD-L1 activity (e.g., binding to PD-1 and/or CD80) are useful for enhancing an anti-tumor immune response. Anti-PD-L1 antibodies are known in the art and described for example in the following US Patent Publications: US20090055944 (BMS/Medarex), which corresponds to WO2007/005874; US2006/0153841 (Dana Farber) corresponding to WO01/14556; US2011/0271358 (Dana Farber); US2010/0203056 (Genentech) issued as U.S. Pat. No. 8,217,149 corresponding to WO2010/077634; US2012/0039906 (INSERM); US20140044738 (Amplimmune) corresponding to WO2012/145493; US20100285039 (John's Hopkins University); and U.S. Pat. No. 8,779,108 (MEDI4736), each of which is incorporated herein by reference.

MEDI4736 is an exemplary anti-PD-L1 antibody that is selective for PD-L1 and blocks the binding of PD-L1 to the PD-1 and CD80 receptors. MEDI4736 can relieve PD-L1-mediated suppression of human T-cell activation in vitro and inhibits tumor growth in a xenograft model via a T-cell dependent mechanism.

Information regarding MEDI4736 (or fragments thereof) for use in the methods provided herein can be found in U.S. Pat. No. 8,779,108, the disclosure of which is incorporated herein by reference in its entirety. The fragment crystallizable (Fc) domain of MEDI4736 contains a triple mutation in the constant domain of the IgG1 heavy chain that reduces binding to the complement component C1q and the Fcγ receptors responsible for mediating antibody-dependent cell-mediated cytotoxicity (ADCC).

MEDI4736 and antigen-binding fragments thereof for use in the methods provided herein comprises a heavy chain and a light chain or a heavy chain variable region and a light chain variable region. In a specific aspect, MEDI4736 or an antigen-binding fragment thereof for use in the methods provided herein comprises a light chain variable region and a heavy chain variable region. In a specific aspect, MEDI4736 or an antigen-binding fragment thereof for use in the methods provided herein comprises a heavy chain variable region and a light chain variable region, wherein the heavy chain variable region comprises the Kabat-defined CDR1, CDR2, and CDR3 sequences shown herein above, and wherein the light chain variable region comprises the Kabat-defined CDR1, CDR2, and CDR3 sequences shown herein above. Those of ordinary skill in the art would easily be able to identify Chothia-defined, Abm-defined or other CDR definitions known to those of ordinary skill in the art. In a specific aspect, MEDI4736 or an antigen-binding fragment thereof for use in the methods provided herein comprises the variable heavy chain and variable light chain CDR sequences of the 2.14H9OPT antibody as disclosed in U.S. Pat. No. 8,779,108, which is herein incorporated by reference in its entirety.

Anti-CTLA-4 Antibodies

Antibodies that specifically bind CTLA-4 and inhibit CTLA-4 activity are useful for enhancing an anti-tumor immune response. Information regarding tremelimumab (or antigen-binding fragments thereof) for use in the methods provided herein can be found in U.S. Pat. No. 6,682,736 (where it is referred to as 11.2.1), the disclosure of which is incorporated herein by reference in its entirety. Tremelimumab (also known as CP-675,206, CP-675, CP-675206, and ticilimumab) is a human IgG₂ monoclonal antibody that is highly selective for CTLA-4 and blocks binding of CTLA-4 to CD80 (B7.1) and CD86 (B7.2). It has been shown to result in immune activation in vitro and some patients treated with tremelimumab have shown tumor regression.

Tremelimumab for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region comprising the amino acid sequences shown herein above and a heavy chain variable region comprising the amino acid sequence shown herein above. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences shown herein above, and wherein the light chain variable region comprises the Kabat-defined CDR1, CDR2, and CDR3 sequences shown herein above. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises the variable heavy chain and variable light chain CDR sequences of the 11.2.1 antibody as disclosed in U.S. Pat. No. 6,682,736, which is herein incorporated by reference in its entirety.

Other anti-CTLA-4 antibodies are described, for example, in US 20070243184. In one embodiment, the anti-CTLA-4 antibody is Ipilimumab, also termed MDX-010; BMS-734016.

Antibodies

Antibodies that selectively bind CTLA-4, PD-1, or PD-L1 and inhibit the binding or activation of PD-1 and/or PD-L1 are useful in the methods of the invention.

In general, antibodies can be made, for example, using traditional hybridoma techniques (Kohler and Milstein (1975) Nature, 256: 495-499), recombinant DNA methods (U.S. Pat. No. 4,816,567), or phage display performed with antibody, libraries (Clackson et al. (1991) Nature, 352: 624-628; Marks et al. (1991) J. Mol. Biol., 222: 581-597). For other antibody production techniques, see also Antibodies: A Laboratory Manual, eds. Harlow et al., Cold Spring Harbor Laboratory, 1988. The invention is not limited to any particular source, species of origin, method of production.

Intact antibodies, also known as immunoglobulins, are typically tetrameric glycosylated proteins composed of two light (L) chains of approximately 25 kDa each and two heavy (H) chains of approximately 50 kDa each. Two types of light chain, designated as the λ chain and the κ chain, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2.

The subunit structures and three-dimensional configurations of different classes of immunoglobulins are well known in the art. For a review of antibody structure, see Harlow et al., supra. Briefly, each light chain is composed of an N-terminal variable domain (VL) and a constant domain (CL). Each heavy chain is composed of an N-terminal variable domain (VH), three or four constant domains (CH), and a hinge region. The CH domain most proximal to VH is designated as CHL The VH and VL domains consist of four regions of relatively conserved sequence called framework regions (FR1, FR2, FR3, and FR4), which form a scaffold for three regions of hypervariable sequence called complementarity determining regions (CDRs). The CDRs contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L1, L2, and L3, accordingly. CDR3 and, particularly H3, are the greatest source of molecular diversity within the antigen-binding domain. H3, for example, can be as short as two amino acid residues or greater than 26.

The Fab fragment (Fragment antigen-binding) consists of the VH-CH1 and VL-CL domains covalently linked by a disulfide bond between the constant regions. To overcome the tendency of non-covalently linked VH and VL domains in the Fv to dissociate when co-expressed in a host cell, a so-called single chain (sc) Fv fragment (scFv) can be constructed. In a scFv, a flexible and adequately long polypeptide links either the C-terminus of the VH to the N-terminus of the VL or the C-terminus of the VL to the N-terminus of the VH. Most commonly, a 15-residue (Gly4Ser)3 peptide (SEQ ID NO: 27) is used as a linker but other linkers are also known in the art.

Antibody diversity is a result of combinatorial assembly of multiple germline genes encoding variable regions and a variety of somatic events. The somatic events include recombination of variable gene segments with diversity (D) and joining (J) gene segments to make a complete VH region and the recombination of variable and joining gene segments to make a complete VL region. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells undergo somatic mutation.

Based on the estimated number of germline gene segments, the random recombination of these segments, and random VH-VL pairing, up to 1.6×107 different antibodies could be produced (Fundamental Immunology, 3rd ed., ed. Paul, Raven Press, New York, N.Y., 1993). When other processes which contribute to antibody diversity (such as somatic mutation) are taken into account, it is thought that upwards of 1×1010 different antibodies could be potentially generated (Immunoglobulin Genes, 2nd ed., eds. Jonio et al., Academic Press, San Diego, Calif., 1995). Because of the many processes involved in antibody diversity, it is highly unlikely that independently generated antibodies will have identical or even substantially similar amino acid sequences in the CDRs.

The sequences of exemplary anti-CTLA-4, anti-PD-L1 and/or anti-PD-1 CDRs are provided herein. The structure for carrying a CDR will generally be an antibody heavy or light chain or a portion thereof, in which the CDR is located at a location corresponding to the CDR of naturally occurring VH and VL. The structures and locations of immunoglobulin variable domains may be determined, for example, as described in Kabat et al., Sequences of Proteins of Immunological Interest, No. 91-3242, National Institutes of Health Publications, Bethesda, Md., 1991.

Antibodies of the invention (e.g., anti-CTLA-4, anti-PD-L1 and/or anti-PD-1) may optionally comprise antibody constant regions or parts thereof. For example, a VL domain may have attached, at its C terminus, antibody light chain constant domains including human Cκ or Cλ chains. Similarly, a specific antigen-binding domain based on a VH domain may have attached all or part of an immunoglobulin heavy chain derived from any antibody isotope, e.g., IgG, IgA, IgE, and IgM and any of the isotope subclasses, which include but are not limited to, IgG1 and IgG4.

One of ordinary skill in the art will recognize that the antibodies of this invention may be used to detect, measure, and inhibit proteins that differ somewhat from CTLA-4, PD-L1 and PD-1. The antibodies are expected to retain the specificity of binding so long as the target protein comprises a sequence which is at least about 60%, 70%, 80%, 90%, 95%, or more identical to any sequence of at least 100, 80, 60, 40, or 20 of contiguous amino acids described herein. The percent identity is determined by standard alignment algorithms such as, for example, Basic Local Alignment Tool (BLAST) described in Altshul et al. (1990) J. Mol. Biol., 215: 403-410, the algorithm of Needleman et al. (1970) J. Mol. Biol., 48: 444-453, or the algorithm of Meyers et al. (1988) Comput. Appl. Biosci., 4: 11-17.

In addition to the sequence homology analyses, epitope mapping (see, e.g., Epitope Mapping Protocols, ed. Morris, Humana Press, 1996) and secondary and tertiary structure analyses can be carried out to identify specific 3D structures assumed by the disclosed antibodies and their complexes with antigens. Such methods include, but are not limited to, X-ray crystallography (Engstom (1974) Biochem. Exp. Biol., 11:7-13) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

Derivatives

Antibodies of the invention (e.g., anti-CTLA-4, anti-PD-L1 and/or anti-PD-1) may include variants of these sequences that retain the ability to specifically bind their targets. Such variants may be derived from the sequence of these antibodies by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described in Antibody Engineering, 2nd ed., Oxford University Press, ed. Borrebaeck, 1995. These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid.

Derivatives and analogs of antibodies of the invention can be produced by various techniques well known in the art, including recombinant and synthetic methods (Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany).

In one embodiment, a method for making a VH domain which is an amino acid sequence variant of a VH domain of the invention comprises a step of adding, deleting, substituting, or inserting one or more amino acids in the amino acid sequence of the presently disclosed VH domain, optionally combining the VH domain thus provided with one or more VL domains, and testing the VH domain or VH/VL combination or combinations for specific binding to the antigen. An analogous method can be employed in which one or more sequence variants of a VL domain disclosed herein are combined with one or more VH domains.

Analogous shuffling or combinatorial techniques are also disclosed by Stemmer (Nature (1994) 370: 389-391), who describes the technique in relation to a β-lactamase gene but observes that the approach may be used for the generation of antibodies.

In further embodiments, one may generate novel VH or VL regions carrying one or more sequences derived from the sequences disclosed herein using random mutagenesis of one or more selected VH and/or VL genes. One such technique, error-prone PCR, is described by Gram et al. (Proc. Nat. Acad. Sci. U.S.A. (1992) 89: 3576-3580).

Another method that may be used is to direct mutagenesis to CDRs of VH or VL genes. Such techniques are disclosed by Barbas et al. (Proc. Nat. Acad. Sci. U.S.A. (1994) 91: 3809-3813) and Schier et al. (J. Mol. Biol. (1996) 263: 551-567).

Similarly, one or more, or all three CDRs may be grafted into a repertoire of VH or VL domains, which are then screened for an antigen-binding fragment specific for CTLA-4, PD-1 or PD-L1.

A portion of an immunoglobulin variable domain will comprise at least one of the CDRs substantially as set out herein and, optionally, intervening framework regions from the scFv fragments as set out herein. The portion may include at least about 50% of either or both of FR1 and FR4, the 50% being the C-terminal 50% of FR1 and the N-terminal 50% of FR4. Additional residues at the N-terminal or C-terminal end of the substantial part of the variable domain may be those not normally associated with naturally occurring variable domain regions. For example, construction of antibodies by recombinant DNA techniques may result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below.

A skilled artisan will recognize that antibodies of the invention may comprise antigen-binding fragments containing only a single CDR from either VL or VH domain. Either one of the single chain specific binding domains can be used to screen for complementary domains capable of forming a two-domain specific antigen-binding fragment capable of, for example, binding to two of CTLA-4, PD-L1 and PD-1.

Antibodies of the invention (e.g., anti-PD-L1 and/or anti-PD1) described herein can be linked to another functional molecule, e.g., another peptide or protein (albumin, another antibody, etc.). For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337. The antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

The disclosed antibodies may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain glycosylation site consensus sequences known in the art. Another means of increasing the number of carbohydrate moieties on the antibodies is by chemical or enzymatic coupling of glycosides to the amino acid residues of the antibody. Such methods are described in WO 87/05330 and in Aplin et al. (1981) CRC Crit. Rev. Biochem., 22: 259-306. Removal of any carbohydrate moieties from the antibodies may be accomplished chemically or enzymatically, for example, as described by Hakimuddin et al. (1987) Arch. Biochem. Biophys., 259: 52; and Edge et al. (1981) Anal. Biochem., 118: 131 and by Thotakura et al. (1987) Meth. Enzymol., 138: 350. The antibodies may also be tagged with a detectable, or functional, label. Detectable labels include radiolabels such as 131I or 99Tc, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

Antibodies, in which CDR sequences differ only insubstantially from those set forth herein are encompassed within the scope of this invention. Typically, an amino acid is substituted by a related amino acid having similar charge, hydrophobic, or stereochemical characteristics. Such substitutions would be within the ordinary skills of an artisan. Unlike in CDRs, more substantial changes can be made in FRs without adversely affecting the binding properties of an antibody. Changes to FRs include, but are not limited to, humanizing a non-human derived or engineering certain framework residues that are important for antigen contact or for stabilizing the binding site, e.g., changing the class or subclass of the constant region, changing specific amino acid residues which might alter the effector function such as Fc receptor binding, e.g., as described in U.S. Pat. Nos. 5,624,821 and 5,648,260 and Lund et al. (1991) J. Immun. 147: 2657-2662 and Morgan et al. (1995) Immunology 86: 319-324, or changing the species from which the constant region is derived.

One of skill in the art will appreciate that the modifications described above are not all-exhaustive, and that many other modifications would obvious to a skilled artisan in light of the teachings of the present disclosure.

Co-Therapy

Treatment of a patient with a solid tumor using a combination of the invention, such as doxorubicin or Doxil and GITR ligand (GITRL) or an OX40 fusion protein, or doxorubicin or Doxil and any one of an anti-CTLA-4 antibody, anti-PD-1 antibody, an anti-PD-L1 antibody, or an antigen-binding fragments thereof as provided herein can result in an additive or synergistic effect. As used herein, the term “synergistic” refers to a combination of therapies (e.g., a combination of doxorubicin or Doxil and GITR ligand (GITRL) or an OX40 fusion protein, or doxorubicin or Doxil and any one of an anti-CTLA-4 antibody, anti-PD-1 antibody, an anti-PD-L1 antibody or antigen binding fragments thereof), which is more effective than the additive effects of the single therapies.

In certain embodiments, synergy is determined by statistical analysis using a Bliss independence model (Zhao et al., J Biomol Screen 2014; 19(5):817-21). The model is described as follows. If the rate of total tumor regression due to drug A alone is rₐ and the rate due to drug B alone is r_{b}, then the expected rate of total tumor regression due to drug A and drug B in combination is r_{Bliss}=rₐ+r_{b}−rₐr_{b} assuming that the two drugs are bliss independent. The difference between the observed total tumor regression rate r_{ab} and the expected rate is defined as the synergy index:

I=r_{ab}−r_{Bliss}

Then the variance of the synergy index can be written as

var(I)=var(r_{ab})+var(r_{Bliss})

Further,

$$var ( {r}_{Bliss} ) = var ( {r}_{a} ) + var ( {r}_{b} ) + var ( {r}_{a} {r}_{b} ) - 2 c ov ( {r}_{a} + {r}_{b} , {r}_{a} {r}_{b} )$$

$$var ( {r}_{a} {r}_{b} ) = var ( {r}_{a} ) + var ( {r}_{b} ) + {r}_{a}^{2} var ( {r}_{b} ) + {r}_{b}^{2} var ( {r}_{a} )$$

$$cov ( {r}_{a} + {r}_{b} , {r}_{a} {r}_{b} ) = {r}_{a} var ( {r}_{b} ) + {r}_{b} var ( {r}_{a} )$$

$$var ( {r}_{ab} ) = \frac{{r}_{ab} ( 1 - {r}_{ab} )}{{n}_{ab}}$$

$$var ( {r}_{a} ) = \frac{{r}_{a} ( 1 - {r}_{a} )}{{n}_{a}}$$

$$var ( {r}_{b} ) = \frac{{r}_{b} ( 1 - {r}_{b} )}{{n}_{b}}$$

where n_{ab}, nₐ, and n_{b} are the respective sample sizes of the combination experiment and two monotherapy experiments. The two drugs are said to be synergistic if

$$\frac{I}{\sqrt{var ( 1 )}} > {Z}_{0.95}$$

where Z_{0.95} is the 95% percentile of standard normal distribution.

A synergistic effect of a combination of therapies (e.g., a combination of doxorubicin or Doxil and GITR ligand (GITRL) or an OX40 fusion protein, or doxorubicin or Doxil and any one of an anti-CTLA-4 antibody, anti-PD-1 antibody, an anti-PD-L1 antibody or antigen binding fragments thereof) permits the use of lower dosages of one or more of the therapeutic agents and/or less frequent administration of said therapeutic agents to a patient with a solid tumor. The ability to utilize lower dosages of therapeutic agents and/or to administer said therapies less frequently reduces the toxicity associated with the administration of said therapies to a subject without reducing the efficacy of said therapies in the treatment of a solid tumor. In addition, a synergistic effect can result in improved efficacy of therapeutic agents in the management, treatment, or amelioration of an solid tumor. The synergistic effect of a combination of therapeutic agents can avoid or reduce adverse or unwanted side effects associated with the use of either single therapy.

In co-therapy, a combination of doxorubicin or Doxil and GITR ligand (GITRL) or an OX40 fusion protein, or doxorubicin or Doxil and any one of an anti-CTLA-4 antibody, anti-PD-1 antibody, an anti-PD-L1 antibody or antigen binding fragments thereof can be optionally included in the same pharmaceutical composition, or may be included in a separate pharmaceutical composition. In this latter case, the pharmaceutical composition comprising doxorubicin or Doxil is suitable for administration prior to, simultaneously with, or following administration of the pharmaceutical composition comprising GITR ligand, OX40 fusion protein, anti-CTLA-4 antibody, anti-PD-1 antibody, an anti-PD-L1 antibody or antigen binding fragments thereof. In certain instances, the doxorubicin or Doxil is administered at overlapping times as GITR ligand, OX40 fusion protein, anti-CTLA-4 antibody, anti-PD-1 antibody, an anti-PD-L1 antibody, or an antigen-binding fragment thereof in a separate composition. MEDI4736 or an antigen-binding fragment thereof and tremelimumab or an antigen-binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

The methods provided herein can decrease or retard tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population. In other embodiments, the methods of the invention increase survival.

Kits

The invention provides kits for enhancing anti-tumor activity. In one embodiment, the kit includes a therapeutic composition containing an effective amount of doxorubicin or Doxil and one or more of an anti-CTLA-4 antibody, anti-PD-1 antibody, anti-PD-L1 antibody, GITR ligand, OX40 fusion protein in unit dosage form.

In some embodiments, the kit comprises a sterile container which contains a therapeutic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for administering the therapeutic combinations of the invention. In particular embodiments, the instructions include at least one of the following: description of the therapeutic agent; dosage schedule and administration for enhancing anti-tumor activity; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. Treatment with Doxil or Doxorubicin in Combination with Checkpoint Inhibitors had Synergistic Anti-Tumor Activity

To test the hypothesis that doxorubicin or Doxil may potentiate anti-tumor effects IMT-C (immune-mediated therapies for cancer) agents, CT26 tumor-bearing Balb/C mice were treated with varying doses of these drugs alone and in combination with anti-mouse PD-1 and anti-CTLA-4 antibodies. These mice were treated in a preventative manner and received treatment prior to any measureable tumor. As this was a preventative study, the dosing of anti-PD-1 and anti-CTLA-4 antibodies was lowered as higher doses in this setting are known to produce strong anti-tumor responses.

A CT26 model was used in several studies. CT26 cells are murine colon cancer cells. CT26 cells were obtained from the ATCC (Manassas, Va.), and were grown with RPMI supplemented with 10% fetal bovine serum. Following receipt, cell lines were re-authenticated using STR-based DNA profiling and multiplex PCR (IDEXX Bioresearch, Columbia, Mo.). Cells were grown in monolayer culture, harvested by trypsinizatin, and implanted subcutaneously into the right flank of 6-8 week old female Balb/C (CT26), C57/Bl6 (MCA205), or 4-6 week athymic female nude mice (Harlan, Indianapolis, Ind.). For the CT26 tumor model, 5×10⁵ cells were implanted in the right flank using a 26-gauge needle. Antibodies were obtained from Anti-PD-1 (RMP1-14), anti-PD-L1 (10F.9G2), anti-CTLA-4 (9D9), and mouse IgG2b control (MPC-11). Mouse OX40 fusion protein (OX40 FP) and Rat IgG2a isotype control antibodies were produced by MedImmune (Gaithersburg, Md.). All antibodies and OX40 FP were dosed via intraperitoneal injection. Doxil (Bluedoor Pharma, Rockville, Md.) and doxorubicin (Henry Schein, Melville, N.Y.) were dosed via intravenous administration. In some studies, isotype controls were administered to mice as a cocktail of rat IgG2a and mouse IgG2b. At the beginning of treatment, mice were either randomized by tumor volume (established-tumor studies) or by body weight (preventative studies). The number of animals per group ranged from between 10-12 animals per group as determined based on sample size calculations using nQuery software. Both tumor and body weight measurements were collected twice weekly and tumor volume calculated using the equation (L×W²)/2, where L and W refers to the length and width dimensions, respectively. Error bars were calculated as standard error of the mean. The general health of mice was monitored daily and all experiments were conducted in accordance to AAALAC and MedImmune IACUC guidelines for humane treatment and care of laboratory animals. Kaplan-Meier statistical analysis was performed using the Log-rank test using GraphPad Prism. The Log-rank (Mantel-Cox) test was used to compare survival curves (Prism 6.03). The Bonferroni method was used to adjust the 0.05 alpha level for multiple comparisons. Reported p-values are two-sided p-values.

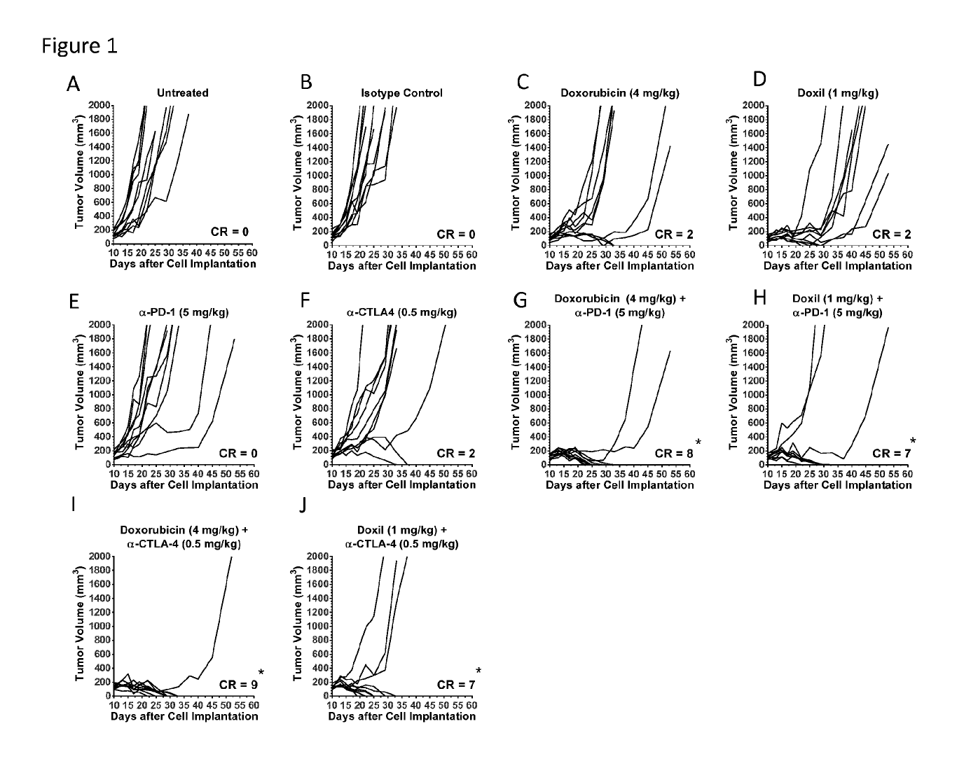

Compared to doxorubicin, Doxil had more potent anti-tumor activity at a 4 mg/kg dose (Table 1). Indeed, all mice treated with Doxil at its MTD (5 mg/kg) had a complete response (CR). A reduced dosage of Doxil at 1 mg/kg had near equivalent anti-tumor activity as doxorubicin at 4 mg/kg (FIGS. 1C and 1D). While anti-PD-1 and anti-CTLA-4 treatment had moderate to low anti-tumor activity as single agents (FIGS. 1E and 1F), both antibodies exhibited synergistic anti-tumor effects when combined with doxorubicin or Doxil (FIGS. 1G-1J). When anti-PD-1 was combined with doxorubicin (4 mg/kg), the number of complete responders increased from 2 to 8 animals (FIG. 1G). Similarly, combination with anti-CTLA-4 increased the number of responders from 2 to 9 animals (FIG. 1I). Similar results were obtained when anti-PD-1 and anti-CTLA-4 were combined with Doxil at 1 mg/kg (FIGS. 1H and 1J). The number of complete responders per group for the entire study is shown at Table 1.

TABLE 1
Doxorubicin or Doxil in combination with PD-1 or CTLA-4 mAbs
produced a high number of cures.
The number of complete responses (CRs) from all of the groups
from the experiment in FIG. 1A-1J is shown. Doxil was more
active than doxorubicin. Doxorubicin or Doxil combined with
PD-1 or CTLA-4 produced a strong anti-tumor response.

| Treatment | No. of CRs |
|---|---|
| Untreated | 0/10 |
| Isotype Controls (5/0.5 mg/kg) | 0/10 |
| Anti-PD-1 (5 mg/kg) | 0/10 |
| Anti-CTLA-4 (0.5 mg/kg) | 0/10 |
| Doxorubicin (4 mg/kg) | 2/10 |
| Doxorubicin (1 mg/kg) | 0/10 |
| Doxil (5 mg/kg) | 10/10 |
| Doxil (4 mg/kg) | 8/10 |
| Doxil (1 mg/kg) | 2/10 |
| Anti-PD-1/Doxorubicin (5/4 mg/kg) | 8/10 |
| Anti-PD-1/Doxorubicin (5/1 mg/kg) | 5/10 |
| Anti-PD-1/Doxil(5/4 mg/kg) | 9/10 |
| Anti-PD-1/Doxil(5/1 mg/kg) | 7/10 |
| Anti-CTLA-4/Doxorubicin (0.5/4 mg/kg) | 9/10 |
| Anti-CTLA-4/Doxorubicin (0.5/1 mg/kg) | 2/10 |
| Anti-CTLA-4/Doxil(0.5/4 mg/kg) | 10/10 |
| Anti-CTLA-4/Doxil(0.5/1 mg/kg) | 7/10 |

These data demonstrate that both doxorubicin and Doxil are synergistic with anti-PD-1 and anti-CTLA-4 antibodies, and indicate this feature to be inherent in doxorubicin itself, as utilization of liposomal doxorubicin had similar synergistic activity with IMT-C as the free drug.

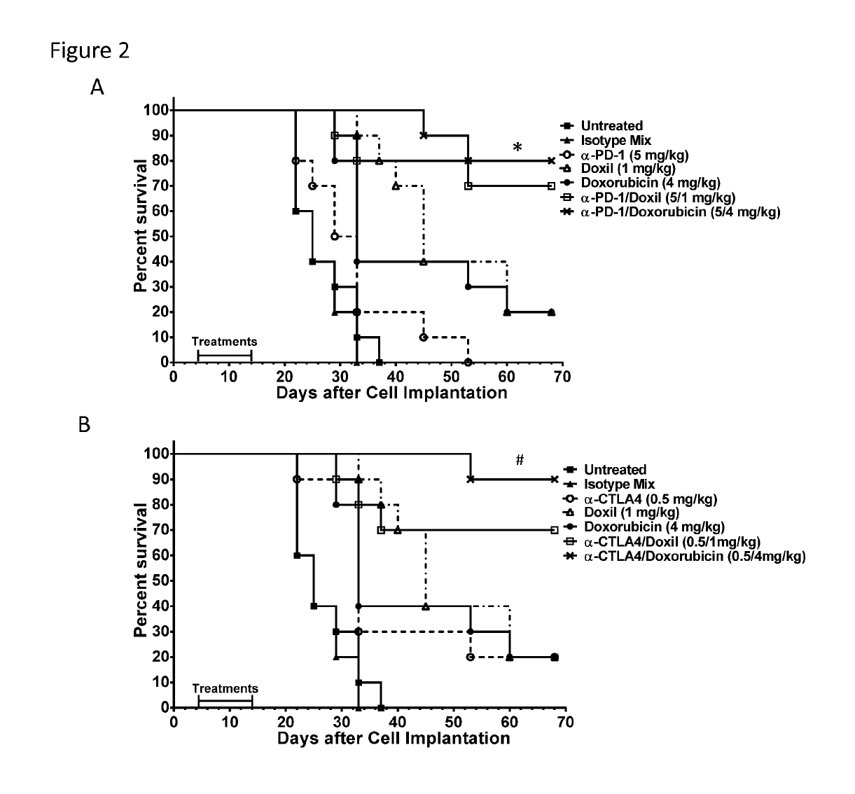

At study completion, the mice treated with the combination of an anti-PD-1 antibody and doxorubicin (4 mg/kg) demonstrated increased survival compared to single-agent therapy (FIG. 2A, p=0.005). Similarly, Doxorubicin (4 mg/kg) or Doxil (1 mg/kg) in combination with anti-CTLA-4 showed improved survival compared to any single agent alone (FIG. 2B, p=0.012). Although not statistically significant, mice treated with Doxil (1 mg/kg)+ either anti-CTLA-4 or anti-PD-1 antibodies also trended toward having longer survival than mice treated with single agent therapy in this study.

### Example 2. Treatment with Doxil Alone or in Combination with Checkpoint Inhibitors Resulted in Tumor-Specific Immunological Memory

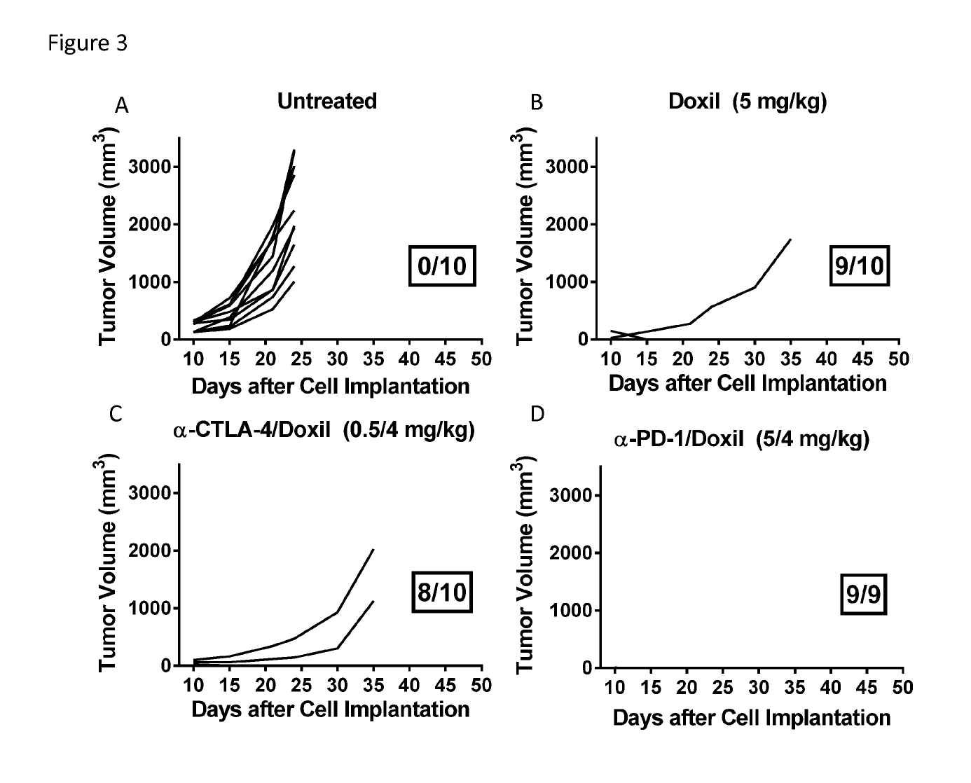

To determine if mice which obtained a complete response with Doxil treatment alone or in combination with anti-PD-1 or anti-CTLA-4 displayed immunological memory, these animals were re-challenged with live CT26 cells 70 days after the initial treatment. While CT26 cells grew in all ten out of ten naïve, untreated mice (FIG. 3A), mice that achieved complete response with Doxil showed widespread tumor rejection with 9 out of 10 mice rejecting tumor (FIG. 3B). Eight out of ten mice treated with Doxil+ anti-CTLA-4 and 9 out of 9 mice with Doxil+ anti-PD-1 rejected tumors (FIGS. 3C and 3D). These results demonstrate that treatment with Doxil as a single-agent, as well as with Doxil in combination with checkpoint inhibitors resulted in tumor-specific immunological memory.

### Example 3. Doxil Anti-Tumor Activity was Observed in Immunocompetent Subjects and Involved T Cells

While both Doxil and doxorubicin were active in a preventative CT26 tumor model, it was also of interest whether these drugs were effective in controlling established CT26 tumors and whether the activity of these drugs was different in the presence of a functional immune system. CT26 cells were implanted into both T cell-deficient athymic nude mice and immunocompetent Balb/C mice and treated with these drugs at their maximally tolerated doses when tumors reached approximately 200 mm³.

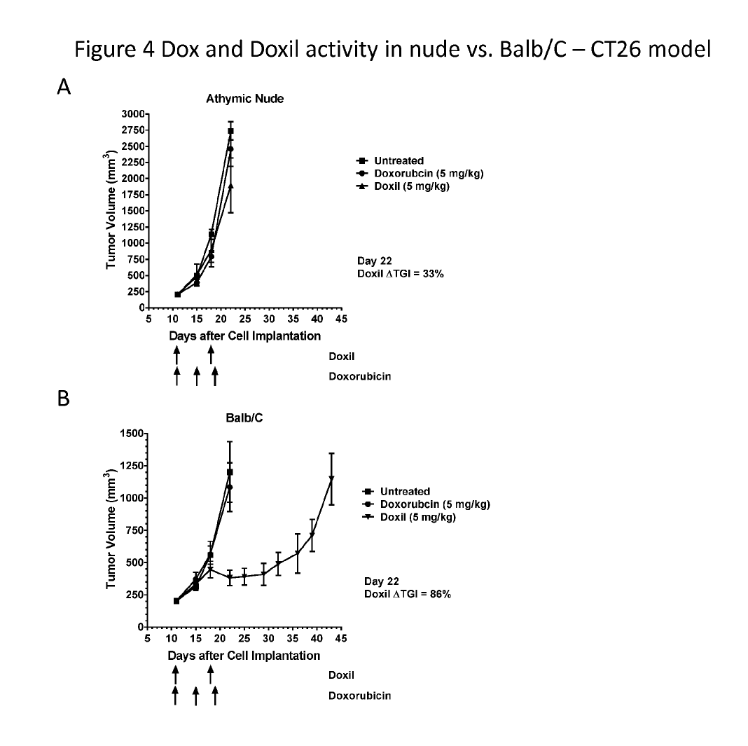

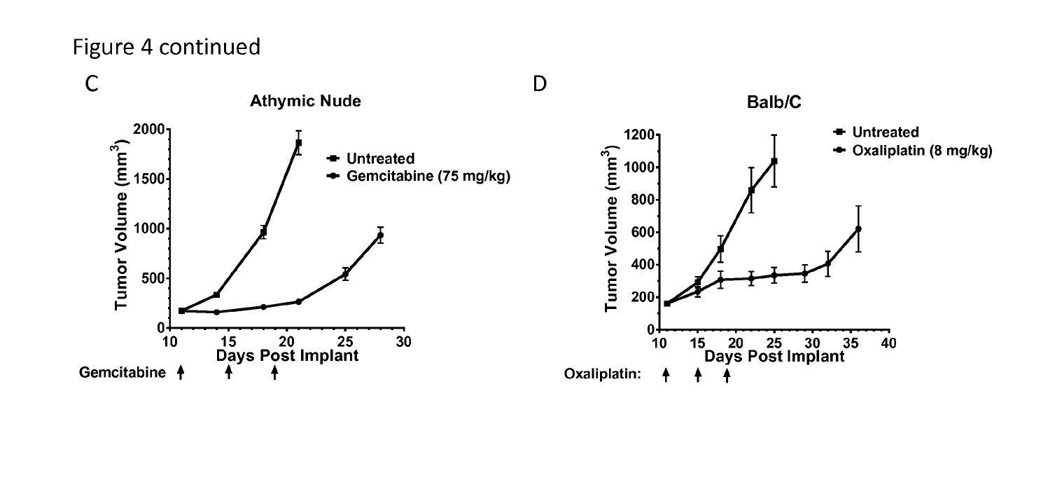

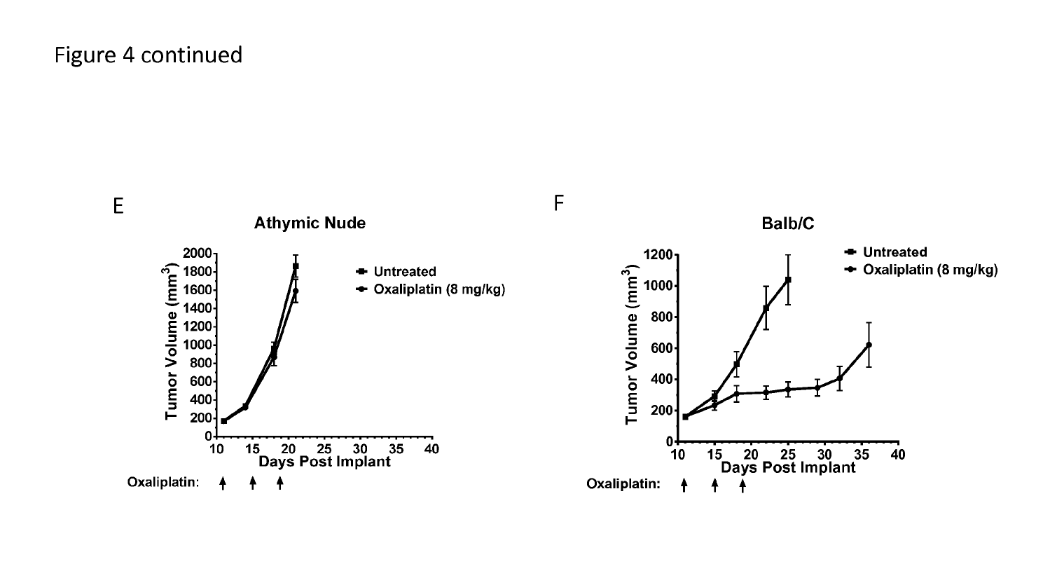

In this experiment, doxorubicin did not elicit anti-tumor activity in either immunocompromised or immunocompetent mice (FIGS. 4A and 4B). In contrast, Doxil treatment showed robust antitumor activity in immunocompetent mice bearing established CT26 tumors (FIG. 4B), but much less activity in immunodeficient mice (FIG. 4A) demonstrating that Doxil activity is increased in the presence of a functional immune system, and likely depends on the presence of T cells.

To assess whether other chemotherapeutic agents could achieve a similar result, immunodeficient and immunocompetent CT26 tumor-bearing mice were administered either oxaliplatin or gemcitabine. Oxaliplatin demonstrated increased antitumor activity in immunocompetent mice (FIG. 4F), as compared to immunodeficient mice (FIG. 4E), consistent with previous reports (Zhao et al. J Biomol Screen 2014; 19(5):817-2). In contrast, gemcitabine had significant anti-tumor activity in both immunodeficient as well as immunocompetent mice (FIGS. 4C and 4D). These results are consistent with prior studies that suggested that certain chemotherapies are efficient inducers of immunological cell death (Obeid et al. Nat Med 2007; 13(1):54-61), and also reveal for the first time that this feature is not held by gemcitabine in vivo.

### Example 4. Doxil is a Booster of Anti-Tumor Activity when Combined with Various Immunomodulatory Agents

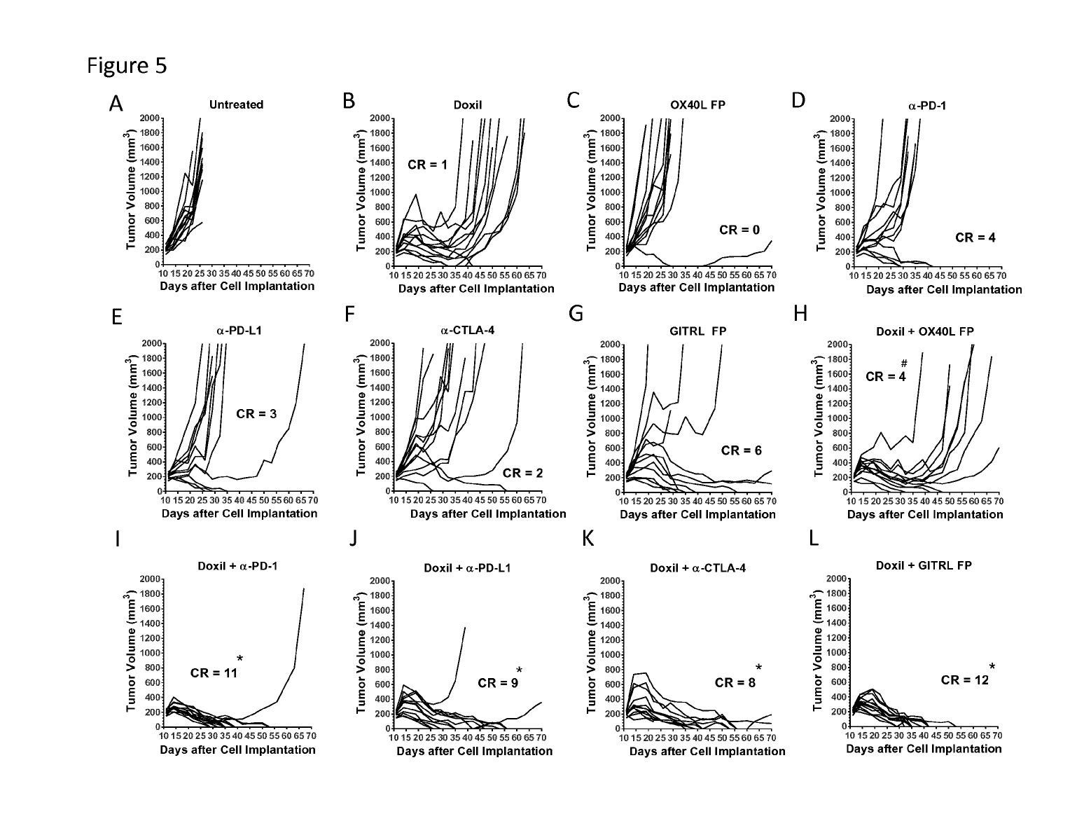

Although the combination of Doxil with anti-PD-1 and anti-CTLA-4 antibodies showed strong anti-tumor effects in the CT26 model (FIGS. 1A-1J), the limitations of the experiment were that it was a preventative study and lower doses of anti-PD-1 and anti-CTLA-4 antibodies were used. To determine whether Doxil remains synergistic with IMT-C in an established-tumor setting, CT26 tumor-bearing mice were treated with Doxil alone and in combination with IMT-C agents targeting CTLA-4 (anti-CTLA-4 (9D9), West Lebanon, N.H.), anti-PD-1 (PD-1 (RMP1-14), West Lebanon, N.H.), PD-L1 (anti-PD-L1 (10F.9G2), West Lebanon, N.H.), OX40 (mouse OX40 fusion protein, MedImmune, Gaithersburg, Md.) and GITR (mouse GITRL ligand fusion protein, MedImmune, Gaithersburg, Md.) all at maximally efficacious doses once tumors were around 200-300 mm³ (FIGS. 5A-5L). Prior studies demonstrated that higher doses of these anti-mouse IMTC-agents did not result in more anti-tumor efficacy at these established tumor volumes.

Doxil treatment resulted in a temporary control of tumor growth, followed by rapid regrowth of tumors, and only one complete response (FIG. 5B). Treatment with OX40 FP, anti-PD-1, anti-PD-L1, anti-CTLA-4 antibodies demonstrated low to moderate activity (FIGS. 5C-5F), with a few complete responders in each group. The combination of Doxil with OX40 FP increased the time to tumor progression compared to single-agent therapy, and a trend toward statistically significant synergy (FIG. 5G). GITR ligand alone produced a more robust response with 6/12 complete responders (FIG. 5G). The combination of Doxil with OX40 FP produced a modest increase in activity compared to either single agent (FIG. 5H).

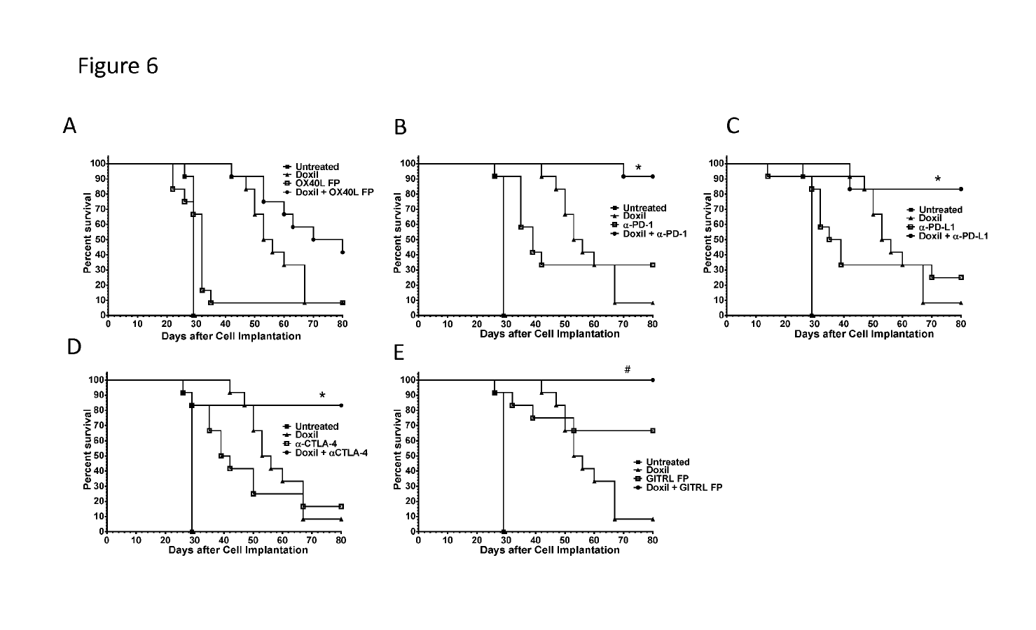

Strikingly, Doxil in combination with anti-PD-1, anti-PD-L1 and anti-CTLA-4 antibodies produced a synergistic increase in the number of complete responders, with 11/12, 9/12, and 8/12 cures, respectively (FIGS. 5H-5J). Remarkably, the combination of Doxil and GITRL FP cured all mice with 12/12 complete responses (FIG. 5L). These experiments demonstrated that Doxil in combination with checkpoint blockers produced dramatic increases in anti-tumor responses, even in established-tumor settings. This was reflected also in Kaplan-Meier survival plots which demonstrated that all mice treated with the combination of Doxil plus PD-1, PD-L1 and CTLA-4 antibodies survived longer than either single agent alone (FIGS. 6A-6E).

### Example 5. Doxil is a Booster of Anti-Tumor Activity when Combined with Multiple Immunomodulatory Agents

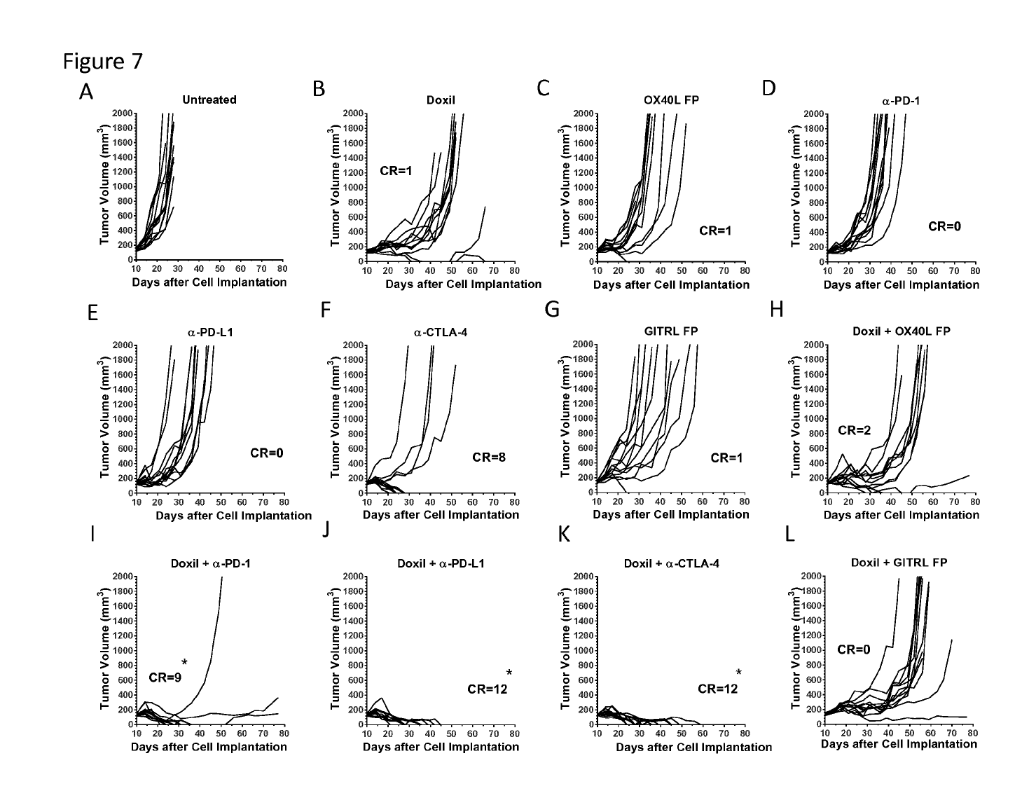

As CT26 is highly sensitive to immunotherapies, it was determined whether Doxil could enhance the activity of immunotherapies in a less sensitive model, MCA205. Doxil, anti-CTLA-4 (9D9, West Lebanon, N.H.); anti-PD-1 (RMP1-14, West Lebanon, N.H.); anti-PD-L1 (10F.9G2, West Lebanon, N.H.); OX40 FP (mouse OX40 fusion protein (MedImmune, Gaithersburg, Md.) and GITRL FP (mouse GITR ligand fusion protein, MedImmune, Gaithersburg, Md.) were dosed at their maximally efficacious doses in established MCA205 tumors starting at a tumor volume between 100-150 mm³ (FIGS. 7A-7L).

MCA205 cells were obtained from Agonox (Portland, Oreg.) and grown in RPMI supplemented with 10% fetal bovine serum. Following receipt, cell lines were re-authenticated using STR-based DNA profiling and multiplex PCR (IDEXX Bioresearch, Columbia, Mo.). MCA205 are fibrosarcoma tumor cells. For the MCA205 tumor model, 2.5×10⁵ cells were implanted. All antibodies and OX40 FP were dosed via intraperitoneal injection. Doxil (Bluedoor Pharma, Rockville, Md.) and doxorubicin (Henry Schein, Melville, N.Y.) were dosed via intravenous administration. In some studies, isotype controls were administered to mice as a cocktail of rat IgG2a and mouse IgG2b. At the beginning of treatment, mice were either randomized by tumor volume (established-tumor studies) or by body weight (preventative studies). The number of animals per group ranged from between 10-12 animals per group as determined based on sample size calculations using nQuery software. Both tumor and body weight measurements were collected twice weekly and tumor volume calculated using the equation (L×W²)/2, where L and W refers to the length and width dimensions, respectively. Error bars were calculated as standard error of the mean. The general health of mice was monitored daily and all experiments were conducted in accordance to AAALAC and MedImmune IACUC guidelines for humane treatment and care of laboratory animals. Kaplan-Meier statistical analysis was performed using the Log-rank test using GraphPad Prism. The Log-rank (Mantel-Cox) test was used to compare survival curves (Prism 6.03). The Bonferroni method was used to adjust the 0.05 alpha level for multiple comparisons. Reported p-values are two-sided p-values.

In this model, Doxil temporarily controlled tumor growth, however most of the tumors re-grew (FIG. 7B). One mouse did achieve a complete response in the Doxil group. OX40 FP, PD-1, GITRL FP and PD-L1 antibodies were minimally active in this model, with some delay in tumor progression, but only one complete response in the OX40 FP group (FIG. 7C-7G). Treatment with an anti-CTLA-4 antibody produced a moderate response with 8/12 mice achieving complete response. For the combination treatments, combining Doxil with an OX40 FP agonist did not delay tumor growth more than Doxil alone, and also did not provide a significant increase in complete responses (FIG. 7H).

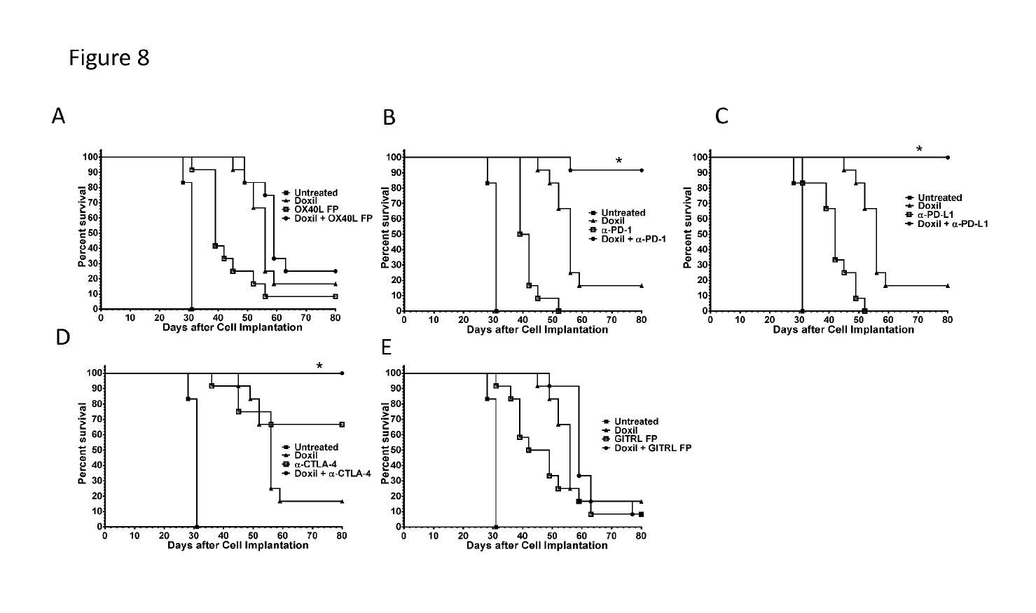

In contrast, Doxil in combination with antibodies to checkpoint inhibitors PD-1, PD-L1 and CTLA-4 produced striking responses with 9/12, 12/12, and 12/12 mice achieving complete response, respectively (FIGS. 7I-7K). These results indicate that Doxil enhances antitumor effects of immunotherapies in models which are not as sensitive to immunotherapies as single agents. Increased survival was observed in mice treated with Doxil+ anti-PD-1, anti-PD-L1 and anti-CTLA-4 antibodies compared to Doxil treatment in this study (FIGS. 8A-8E).

### Example 6 Doxil Decreased Tumor Tregs, Induced Cytotoxic T Cell Expansion, and Activated Mature DCs in Tumors

A mechanistic study was performed in order to elucidate any effects of Doxil on immune cell populations in vivo. MCA205-tumor bearing mice were treated with Doxil, α-PD-L1 antibody or the combination and tumors and blood were harvested. MCA205 cells (2.5×10⁵) were implanted in the right flank of 6-8 week old C57/Bl6 female mice. When tumors reached an average of ˜250 mm³, mice were randomized in groups of 6 and were dosed with Doxil (5 mg/kg); OX40 FP (20 mg/kg); α-PD-L1 (20 mg/kg) or a combination of Doxil with OX40 FP or α-PD-L1 (Day 0). A second dose of OX40 FP and α-PD-L1 was given on Day 3, and Doxil again on Day 7. On Day 8 all mice were euthanized and tissues were collected from mice. Red blood cells were lysed with ACK solution (Life Tech, Carlsbad, Calif.). Tumors were cut into 2 mm³ pieces and digested for 20 min at 37° C. with a 200 units/mL Collagenase type 3 (Worthington, Lakewood, N.J.) and 0.25 mg/mL DNase (Sigma-Aldrich, St. Louis, Mo.). One to two million cells were loaded per well in a 96 well plate and stained Live Dead Blue (Life Tech, Carlsbad, Calif.) and stained with antibodies to CD111b (BD Clone M1/70), CD11c (Biolegend Clone n418), CD80 (Biolegend Clone 16-10A1), Ly6G (Biolegend Clone 1A8), Ly6C (Biolegend HK1.4), CD45 (Ebioscience Clone 30-F11), MHC-II (Biolegend Clone M5/114.15.2), CD4 (Biolegend Clone RM4-5), CD8 (BD Clone RPA-T8), and FOXP3 (Ebioscience Clone FJK-16S) in FACS buffer (PBS+0.5% FBS and 2 mm EDTA). For FOXP3 detection, a FOXP3 transcription kit was used (Ebioscience, San Diego, Calif.). Cells were stained at 4° C. for 20 minutes, washed, and fixed with 4% Paraformaldehyde. Sample data were acquired on a BD Fortessa (BD, San Jose, Calif.). Data were analyzed using Flowjo (Treestar, Ashland, Oreg.).

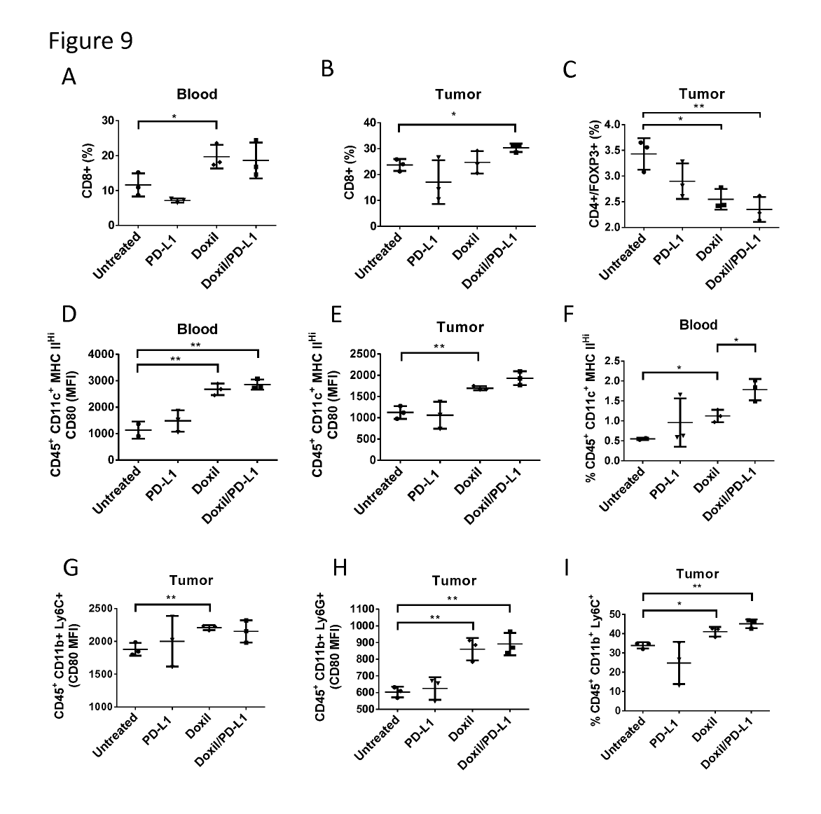

In MCA205-tumor bearing mice were treated with Doxil, α-PD-L1 antibody or the combination, Doxil increased the percent of CD8+ T cells in the blood, and that the combination of Doxil and P α-D-L1 produced a significant increase in the percent of CD8⁺ T cells in the tumor (FIGS. 9A and 9B). In addition to cytotoxic T-cells, a significant decrease in the amount of tumor infiltrating Tregs was observed as a result of Doxil treatment, which seemed to be further augmented by the combination of Doxil and anti-PD-L1 (FIG. 9C).

To examine the cause of the T-cell changes, the phenotype of the myeloid compartment in blood and tumor was investigated. In both the blood and tumor, Doxil and Doxil+ anti-PD-L1, but not anti-PD-L1 alone, induced the expression of the co-stimulatory molecule CD80 on CD45⁺CD11c⁺MHCII^{hi} cells, which represent mature dendritic cells (FIGS. 9D and 9E). The level of CD80 expression tended to be higher in the combination group compared to Doxil alone. At the same time, Doxil treatment also increased the percent of CD45⁺CD11c⁺MHCII^{hi} cells in the blood, which was further significantly increased when combined with anti-PD-L1 (FIG. 9F). This demonstrates that Doxil not only increased the level of CD80 on mature dendritic cells, but also induced expansion of these cells. Interestingly, the effect of Doxil-induced upregulation of CD80 was also observed on CD45⁺CD11b⁺Ly6c⁺ monocytic MDSC and CD45⁺CD11b⁺Ly6G⁺ granulocytic MDSCs in the tumor (FIGS. 9G and 9H). Doxil as well as Doxil+ anti-PD-L1 also increased the percent of CD45⁺CD11b⁺Ly6c⁺ cells in the tumor (FIG. 9I). In summary, these results demonstrated that in vivo, Doxil decreased tumor Tregs, induced cytotoxic T cell expansion, and activated mature DCs in tumors. These findings are consistent with and may provide an explanation for the profound anti-tumor effects that Doxil had in combination with anti-PD-L1, and potentially other mediators of checkpoint blockade as observed in this study as well.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of increasing anti-tumor activity in a subject, the method comprising administering doxorubicin or polyethylene glycol coated liposome encapsulated form of doxorubicin and an immunomodulatory agent selected from the group consisting of an anti-PD-1 antibody, anti-PD-L1 antibody, anti-CTLA-4 antibody, a glucocorticoid-induced TNFR-related gene (GITR) ligand, and an OX40 fusion protein to a subject.

2. A method of increasing an anti-tumor immune response in a subject, the method comprising administering doxorubicin or a polyethylene glycol coated liposome encapsulated form of doxorubicin and an immunomodulatory agent selected from the group consisting of an anti-PD-1 antibody, anti-PD-L1 antibody, anti-CTLA-4 antibody, a GITR ligand, and an OX40 fusion protein to a subject.

3. A method of treating a tumor in a subject, the method comprising administering doxorubicin or polyethylene glycol coated liposome encapsulated form of doxorubicin and an immunomodulatory agent selected from the group consisting of an anti-PD-1 antibody, anti-PD-L1 antibody, anti-CTLA-4 antibody, a GITR ligand, and an OX40 fusion protein to a subject.

4. The method of claim 1, wherein the polyethylene glycol coated liposome encapsulated form of doxorubicin is Doxil®.

5. The method of claim 1, wherein the anti-PD-L1 antibody is MEDI4736, BMS-936559, or MPDL3280A.

6. The method of any one of claim 5, wherein the anti-PD-L1 antibody is MEDI4736.

7. The method of claim 1, wherein the anti-PD-1 antibody is LOPD 18, nivolumab, pembrolizumab, lambrolizumab, MK-3475, AMP-224, and pidilizumab.

8. The method of claim 7, wherein the anti-PD-1 antibody is LOPD 18.

9. The method of claim 1, wherein the anti-CTLA-4 antibody is tremelimumab or ipilimumab.

10. The method of claim 9, wherein the anti-CTLA-4 antibody is tremelimumab.

11. The method of claim 1, wherein the immunomodulatory agent is a GITR ligand or GITR ligand fusion protein.

12. The method of claim 1, wherein the immunomodulatory agent is an OX40 fusion protein.

13. The method of claim 1, wherein the tumor is a colon carcinoma or sarcoma.

14. The method of claim 1, wherein the method results in an increase in overall survival as compared to the administration of any one of doxorubicin, a polyethylene glycol coated liposome encapsulated form of doxorubicin, an anti-PD-1 antibody, anti-PD-L1 antibody, anti-CTLA-4 antibody, a GITR ligand, and an OX40 fusion protein alone.

15. The method of claim 1, wherein the method induces a tumor-specific immune response.

16. The method of claim 1, wherein doxorubicin or Doxil is administered in combination with an anti-PD-1 antibody.

17. The method of claim 16, wherein the anti-PD-1 antibody is LOPD 18, nivolumab, pembrolizumab, lambrolizumab, MK-3475, AMP-224, or pidilizumab.

18. The method of claim 1, wherein doxorubicin or Doxil is administered in combination with an anti-PD-L1 antibody.

19. The method of claim 18, wherein the anti-PD-L1 antibody is MEDI4736, BMS-936559, or MPDL3280A.

20. The method of claim 1, wherein doxorubicin or Doxil is administered in combination with an anti-CTLA-4 antibody.

21. The method of claim 20, wherein the anti-CTLA-4 antibody is tremelimumab or ipilimumab.

22. The method of claim 1, wherein doxorubicin or Doxil is administered in combination with a GITR ligand or GITR ligand fusion protein.

23. The method of claim 1, wherein doxorubicin or Doxil is administered in combination with an OX40 fusion protein.

24. The method of claim 1, wherein the administration of doxorubicin, a polyethylene glycol coated liposome encapsulated form of doxorubicin, an anti-PD-1 antibody, anti-PD-L1 antibody, anti-CTLA-4 antibody, a GITR ligand, or an OX40 fusion protein is by intravenous infusion.

25. The method of claim 1, wherein the doxorubicin or a polyethylene glycol coated liposome encapsulated form of doxorubicin and the immunomodulatory agent are administered concurrently.

26. The method of claim 1, wherein the doxorubicin or a polyethylene glycol coated liposome encapsulated form of doxorubicin is administered prior to the immunomodulatory agent.

27. The method of claim 1, wherein the immunomodulatory agent is administered prior to the doxorubicin or a polyethylene glycol coated liposome encapsulated form of doxorubicin.

28. The method of claim 1, wherein the subject is a human patient.

29. A kit for increasing anti-tumor activity, the kit comprising doxorubicin or a polyethylene glycol coated liposome encapsulated form of doxorubicin and immunomodulatory agent selected from the group consisting of an anti-PD-1 antibody, anti-PD-L1 antibody, anti-CTLA-4 antibody, a GITR ligand, and an OX40 agonist.

30. The kit of claim 28, wherein the kit further comprises instructions for using the kit in the method of claim 1.

31. A pharmaceutical formulation comprising an effective amount of doxorubicin or a polyethylene glycol coated liposome encapsulated form of doxorubicin and an effective amount of an immunomodulatory agent selected from the group consisting of an anti-PD-1 antibody, anti-PD-L1 antibody, anti-CTLA-4 antibody, a GITR ligand, and an OX40 agonist.

32. The method of claim 31, wherein the anti-CTLA-4 antibody is tremelimumab or ipilimumab.

33. The method of claim 31, wherein the anti-PD-1 antibody is LOPD 18, nivolumab, pembrolizumab, lambrolizumab, MK-3475, AMP-224, or pidilizumab.

34. The method of claim 31, wherein the anti-PD-L1 antibody is MEDI4736, BMS-936559, or MPDL3280A.


SEQUENCE LISTING (27 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 127 aa, Artificial Sequence
  Synthetic polypeptide
QVQLQESGPGLVKPSQTLSLTCTVSGGSISSGAYYWSWIRQHPGKGLEWIGYIYYNGNTYYNPSLRSRVTISVDTSKNQFSLKLSSVTAADTAVYYCVRASDYVWGGYHYFDAFDLWGRGTLVTVSS

SEQ ID NO: 3 - DNA, 381 bp, Artificial Sequence
  Synthetic polynucleotide
caggtgcagctgcaggagtcgggcccaggactggtgaagccttcacagaccctgtccctcacctgcactgtctctggtggctccatcagcagtggtgcttattactggagctggatccgccagcacccagggaagggcctggagtggattgggtacatctattacaatgggaacacgtactacaacccgtccctcaggagtcgagttaccatatcagtagacacgtctaagaaccagttctccctgaagctgagctctgtgactgccgcggacacggccgtctattactgtgtgagagcgtctgattacgtttgggggggttatcattatttcgacgcgttcgacctctggggccggggaaccctggtcaccgtctcctca

SEQ ID NO: 4 - protein, 110 aa, Artificial Sequence
  Synthetic polypeptide
QSVLTQPPSASGTPGQRVTISCSGSNSNIGSNSVNWYQQLPGTAPKLLIYGNNQRPSGVPDRFSGSKSGTSASLAISGLQSEDEADYYCAAWDDSLNGPVFGGGTKVTVL

SEQ ID NO: 5 - DNA, 330 bp, Artificial Sequence
  Synthetic polynucleotide
cagtctgtgctgactcagccaccctcagcgtctgggacccccgggcagagggtcaccatctcttgttctggaagcaactccaacatcggaagtaattctgtaaactggtaccagcagctcccaggaacggcccccaaactcctcatctatggtaataatcagcggccctcaggggtccctgaccgattctctggctccaagtctggcacctcagcctccctggccatcagtgggctccagtctgaggatgaggctgattattactgtgcagcatgggatgacagcctgaatggtccggtattcggcggagggaccaaggtcaccgtccta

SEQ ID NO: 6 - protein, 108 aa, Artificial Sequence
  Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 7 - protein, 121 aa, Artificial Sequence
  Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 8 - protein, 11 aa, Artificial Sequence
  Synthetic polypeptide
GFTFSRRYWMS

SEQ ID NO: 9 - protein, 17 aa, Artificial Sequence
  Synthetic polypeptide
NIKQDGSEKYYVDSVKG

SEQ ID NO: 10 - protein, 12 aa, Artificial Sequence
  Synthetic polypeptide
RASQRVSSSYLA

SEQ ID NO: 11 - protein, 7 aa, Artificial Sequence
  Synthetic polypeptide
DASSRAT

SEQ ID NO: 12 - protein, 9 aa, Artificial Sequence
  Synthetic polypeptide
QQYGSLPWT

SEQ ID NO: 13 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 14 - protein, 139 aa, Artificial Sequence
  Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 15 - protein, 167 aa, Artificial Sequence
  Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 16 - protein, 10 aa, Artificial Sequence
  Synthetic polypeptide
GFTFSSYGMH

SEQ ID NO: 17 - protein, 15 aa, Artificial Sequence
  Synthetic polypeptide
VIWYDGSNKYYADSV

SEQ ID NO: 18 - protein, 16 aa, Artificial Sequence
  Synthetic polypeptide
DPRGATLYYYYYGMDV

SEQ ID NO: 19 - protein, 11 aa, Artificial Sequence
  Synthetic polypeptide
RASQSINSYLD

SEQ ID NO: 20 - protein, 7 aa, Artificial Sequence
  artificial sequence
AASSLQS

SEQ ID NO: 21 - protein, 9 aa, Artificial Sequence
  Synthetic polypeptide
QQYYSTPFT

SEQ ID NO: 22 - protein, 255 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCCWRCRRRPKTPEAASSPRKSGASDRQRRRGGWETCGCEPGRPPGPPTAASPSPGAPQAAGALRSALGRALLPWQQKWVQEGGSDQRPGPCSSAAAAGPCRRERETQSWPPSSLAGPDGVGS

SEQ ID NO: 23 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 24 - protein, 410 aa, Artificial Sequence [sequence omitted]

SEQ ID NO: 25 - protein, 6 aa, Mus sp.
VAYEEL

SEQ ID NO: 26 - protein, 6 aa, Homo sapiens
TEYATI

SEQ ID NO: 27 - protein, 15 aa, Artificial Sequence
  Synthetic polypeptide
GGGGSGGGGSGGGGS